(19) United States
(12) Patent Application Publication
(10) Pub. No.: US 2007/0025986 A1
(43) Pub. Date: Feb. 1, 2007

(54) Modulation of HCN channels by second messengers

(76) Inventors: Keri J. Fogle, New York, NY (US); Gareth R. Tibbs, New York, NY (US)

Correspondence Address: BAKER & BOTTS L.L.P., 30 ROCKEFELLER PLAZA, 44TH FLOOR, NEW YORK, NY, 10112-4498, US

(21) Appl. No.: 11/375,979

(22) Filed: Mar. 15, 2006

Related U.S. Application Data
(63) Continuation of application No. PCT/US04/43434, filed on Dec. 23, 2004.
(60) Provisional application No. 60/532,841, filed on Dec. 23, 2003.

(51) Int. Cl. A61K 39/395; A61K 31/366; A61K 31/353; A61K 31/7048

(52) U.S. Cl. 424/143.1; 530/388.22; 514/27; 514/453; 514/456

(57) ABSTRACT

The present invention relates to modulation of the activation of hyperpolarization-activated cyclic-nucleotide-sensitive cation non-selective (HCN) channels by second messengers. The present invention provides methods for modulating the activation of HCN channels, methods for the treatment and prevention of disorders associated with abnormality in HCN channel functions, and methods of screening for compounds that modulate the activation of HCN channels. The present invention further encompasses compounds that modulate the activation of HCN channels and pharmaceutical compositions comprising such compounds.

### CROSS REFERENCE TO RELATED APPLICATION

This application claims the benefit of U.S. Provisional Application No. 60/532,841 filed on Dec. 23, 2003.

### FIELD OF THE INVENTION

The present invention is directed to mechanisms of regulating a particular subclass of ion channels—the hyperpolarization-activated cyclic-nucleotide-sensitive cation non-selective (HCN) pacemaker channels—through second-messenger systems (e.g., lipid and protein kinases).

### BACKGROUND OF THE INVENTION

A. HCN Channels

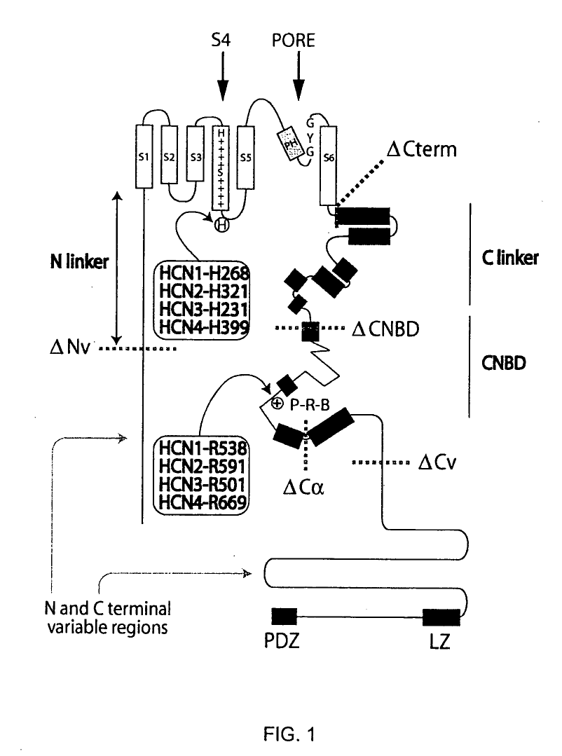

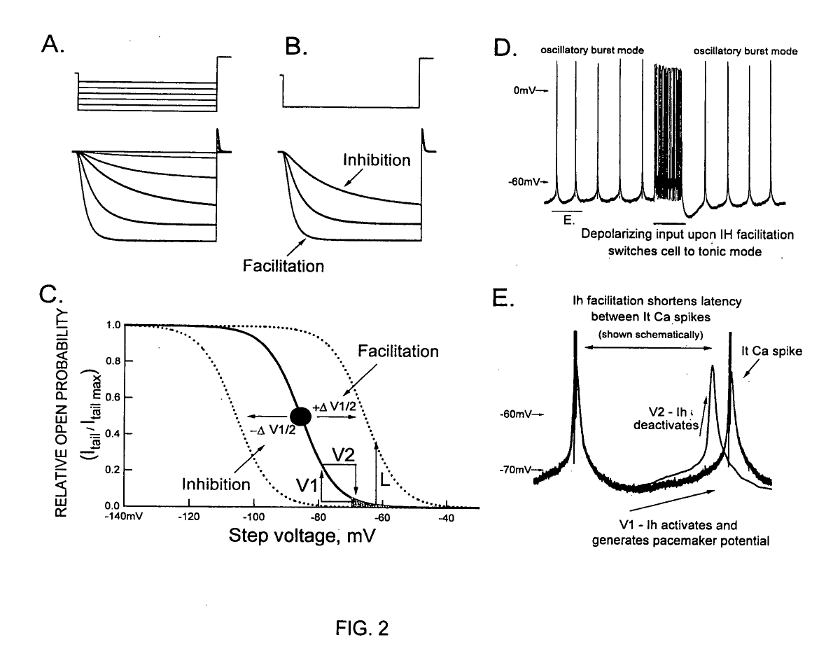

The heartbeat, the conscious/unconscious transition and the brain's processing of sensory information into a coherent representation of the external world are examples of higher order phenomena dependent on the generation and termination of rhythmic firing patterns. Pacemaker channels (FIG. 1) are molecular drives that give rise to, and regulate, such rhythmic activity through sensitivity of their voltage-dependent activation to second messenger regulation (e.g. FIG. 2). Pacemaker channels also serve to set the resting membrane properties of many cells and are, therefore, crucial in determining the fidelity of sensory and motor processing in the central nervous system (e.g. FIG. 2).

Native pacemaker channels (termed I_{Q}, I_{F} or I_{H}) are found in cardiac cells and in both peripheral and central neurons and are encoded by the HCN gene family (FIG. 1). HCN genes are found in mammal, fruit fly, silk moth, and sea urchin. There are four known HCN genes in mammal, HCN1-HCN4, which are members of the voltage-gated K channel superfamily (Santoro et al, 1997, Proc. Natl. Acad. Sci. USA 94: 14815-14820; Santoro et al, 1998, Cell 93:717-729; Ludwig et al, 1998, Nature 393: 587-591). Typically, an HCN channel consists of a core transmembrane segment and a cNMP-binding domain (CNBD) motif.

Recent studies have suggested that changes in HCN isoform expression and/or regulation may underlie several major pathologies including neuropathic pain (Chaplan et al, 2003, J. Neurosci 23: 1169-1178), epilepsy (McCormick et al, 2001, Annu Rev Physiol 63: 815-846), changes associated with hypertrophied or failing heart (Cerbai et al, 2001, J. Mol. Cell Cardiol 33: 441-448.), and other disorders such as schizophrenia (Stopkova et al, 2003, Am. J. Med. Genet. B. Neuropsychiatr. Genet. 123: 50-58), motor learning dysfunction (Nolan et al, 2003, Cell 115: 551-564), and sick sinus syndrome (Schulze-Bahr et al, J. Clin. Invest., 2003, 111: 1537-45). These data suggest HCN channels may be clinically relevant targets for therapeutic intervention in a number of disorders.

However, given the high homology between HCN isoforms and their widespread distribution [7], therapeutic targeting of native pacemaker channels will likely require tissue, as well as isoform, specific targeting. At present the only agents that target HCN channels are a class of organic channel blocking molecules, such as ZD-7288 (Chaplan et al, supra). These compounds were generated as cardiac anti-arrhythmic agents but had no cardiovascular therapeutic utility. Not only did such drugs fail to distinguish between isoforms, they showed no cellular or system discrimination eliciting CNS and visual side effects [37-39]. Moreover, conditions where an up-regulation of channel activity could be therapeutically relevant are not open to such a class of ligands. An alternative strategy—that may permit tissue and isoform specific control of native pacemaker channel function—would be to target second messenger cascades and coupled upstream receptors. A survey of the available literature indicates several candidate pathways.

Many studies have reported receptor-mediated modulation of the voltage-dependent activation of pacemaker channels (See Table 1). The best-described and molecularly understood form of regulation is that mediated via direct binding of cAMP to the channel protein and it is clear from the results summarized in Table 1, that many transmitter and modulator systems alter pacemaker gating by altering the activity of adenylate cyclase. However, it is also clear that the responses of many receptors are unlikely to be mediated via changes in cAMP. Many of these “non-cAMP” receptor-coupled alterations in pacemaker activity may involve activation of either phospholipase C (PLC; β or γ) or a PLC-independent role of tyrosine kinase activation.

The present invention provides insight into how phospholipase C and tyrosine kinase signaling cascades control pacemaker channels and suggest the underlying mechanisms by which many G-protein coupled receptors (GPCR) and trophic factors can exert influence over cellular excitability in physiological and pathological states. The present invention permits novel and specific therapeutic targeting of the activation status of these critical molecules.

Table 1. Receptor Regulation of Native Pacemaker Channels

Symbol code: ↓, inhibition of activity, hyperpolarizing shift in I_{H}V1/2; ↑, enhancement of activity, depolarizing shift in I_{H}V1/2; ˜, no effect; Blank, not determined. Where an entry is not qualified with a numeric value of the shift in I_{MAX} or the V_{1/2} the change was present but not quantifiable. In each of these cases the basis of the second-messenger cascade is suggested by the consensus coupling for that receptor family and is shown in the “coupling” column.

CELL TYPES: AT: atrial myocyte; ECM: Embryonic cardiomyocytes; PF: cardiac purkinje fibre; SA: sinoatrial node. ADn: Thalamic anterior dorsal relay neurons; BC: cerebllar basket cell; BSMN: Brainstem motor neurons; CA1: CA1 pyramidal neurons or unidentified CA1 cells; CMN: crustacean motor neurons [24]; CP: Cerebellar purkinje neurons; DRG: Dorsal root ganglion neurons; FM: Facial motor neurons; FS: facial/spinal motor neurons; HG: Hypoglossal motor neurons; LG: Lateral geniculate thalamic relay neurons; MP: Mesopontine cholinergic; MTNB: medial nucleus of the trapezoid body; NR: Nucleus raphe magnus; ORN: Olfactory receptor neurons; PBC: Pre-botzinger complex; PG: Pyloric ganglion motor neurons; PH: Prepositus Hyperglossi neurons; PVN: Hypothalamic paraventricular neurons; ROD: Rod photoreceptors; SNPC: Substantia nigra pars compacta; SNZC: Substantia nigra zona compacta; SO: CA1 stratum oriens-alveus interneuron; ST: Solitary tract neurons; TR: thalamic relay; TG: Trigeminal ganglion; NG: Nodose ganglion; VT: Ventral tegmental neurons.

TABLE 1

| RECEPTOR SYSTEM | | | | IH MODULATION | |
|---|---|---|---|---|---|
| | | AGONIST | COUPLING | | |
| CLASS | SUBTYPE | (antagonized) | (inhibitors) | Δ IMAX | Δ V1/2 |

A. NON-PEPTIDERGIC RECEPTOR REGULATION OF NATIVE PACEMAKER CHANNELS

| | | | | | | | |
|---|---|---|---|---|---|---|---|
| ADRENERGIC | β | NorE, Isoproterenol, | ↑AC-Gs | ˜ | ˜SA; +90% AT; +65% | ↑ | SA; +11 mV AT; +5 mV TR; |
| | | isoprenaline | | ↑ | ECM; +31% TR; BC; | | +6 mV BC; SO; +12 mV |
| | | (propanolol, atenolol) | | | +30% SO; ˜MTNB; | | MTNB; |
| | α1/2 | Clonidine (yohimbine, | ↓AC-Gi MAPK, | ↓ | HG, −40% DRG | ↓ | −4 mV HG |
| | | idazoxan) | PLC, PLA2, PLD | i | | | |
| CHOLINERGI | m1/3/5 | Acetylcholine, | ↑PLCβ-Gq/11 | ↑ | CA1; +43% LGN | ↑ | CA1; +5 mV LGN |
| C | | carbachol | | ii | | | |
| | m2/4 | Acetylcholine, | ↓AC-Gi/o | ˜ | ˜SA, PF, −41% ECM | ↓ | −8 mV SA, PF |
| | | carbachol | | ↓ | | | |
| SEROTONIN | 5HT | 5-HT (methysergide) | | ˜ | +20% TR; +30% PH; FS; | ↑ | +5 mV TR; ES; +10 mV CMN; |
| | | | | ↑ | +20% CMN ˜BSMN | | +6 mV BSMN |
| | | | | iii | | | |
| | 5HT1 (not | 5-HT | ↓AC-Gi, ↑PLC | ↓ | −50% CA1 | ↓ | −5 mV CA1 |
| | C) | | | | | | |
| | 5HT1 (not | 5-HT, 8-OH-DPAT | ↓AC-Gi, ↑PLC | ↑ | +100% ST | | nd |
| | C) | (NAN-190) | | | | | |
| | 5HT2 | 5-HT, DOI | ↑PLCβ-Gq/11. | ↓ | CP; −40% VT | ↓ | −8 mV CP; −7m VT |
| | | | ↑PLA2 | iv | | | |
| | 5HT2 | 5-HT, DOI | ↑PLCβ-Gq/11. | ↑ | +25% FM; +38% VT | nd | |
| | | | ↑PLA2 | | | | |
| | 5HT4/6/7 | 5-HT, 5CT (spiperone) | ↑AC-Gs | ˜ | +50% CA1; ˜DRG | ↑ | +6 mV DRG; +15 mV ADn; |
| | | | | ↑ | | | +5 mV CA1 |
| DOPAMINE | D1/5 | Dopamine | ↑AC | nd | | ↑ | +20 mV PG |
| | D2 (D4) | Dopamine, quinpirole | ↓AC | ↓ | −20% VT; −50% ORN; | ↓ | −9 mV ORN; −17 mV ROD |
| | | (sulpiride) | | v | −30% ROD | | |
| ADENOSINE | A1 | Adenosine, N6-CPA, | ↓AC | nd | | ↓ | −6 mV SA; LG; −7 mV |
| | | N6-CHA (8-PST, | | | | | DRG; MP; But see |
| | | DPCPX) | | | | | |
| HISTAMINE | H1/2/3 | Histamine (tiotidine) | ↑AC-Gs; H1: | nd | | ↑ | LG |
| | | | ↑PLC H1 | | | | |
| GABAB | | Baclofen | ↓AC | ↓ | −20% VT; −11% SNZC | ˜ | VT, SNZC |
| | | | | vi | | | |

B. PEPTIDERGIC RECEPTOR REGULATION OF NATIVE PACEMAKER CHANNELS

| | | | | | | | |
|---|---|---|---|---|---|---|---|
| NEUROTROPHIN | TrkA/B/C | BDNF | ↑PTK: ↑PLCγ | ↓ | −46% PBC | ↓ | −17 mV PBC |
| | P75 | | | | | | |
| EGF | EGF | EGF | ↑PTKs (gen); | ↑ | +23% SA | ˜ | SA |
| | | | ↑PLCγ; ↑PLA2; | | | | |
| | | | MAPK:PI3K | | | | |
| BRADYKININ | B½ | Bradykinin | ↑PLCβ- | ↑↓ | TG | nd | |
| | | | Gq/11.PLA2 | nd | | | |
| NEUROTENSIN | NT½ | Neurotensin; OAG | ↑PLCβ/PKC | ↓ | −49% SNPC | ˜ | SNPC |
| | | | (staurosporine; | | | | |
| | | | PKC19-31) | | | | |
| NEUROMEDIN | NMU ½ | Neuromedin | ↑PLCβ | ↑ | +10% PVN | ↑ | +11 mV PVN |
| VIP | PAC1 | VIP/PACAP38 | ↑AC | ↑ | +30% TR | ↑ | +7 mV TR |
| ANGIOTENSIN | AT½ | AT-II (losartan) | ↑PLCγ; ↑↓AC | nd | | ↑ | +8m PVN |
| EICOSANOIDS | EP | PGE2 | ↑AC, ↑PKC | ↑ | +18% NG, TG | ↑ | +6 mV NG, TG |
| OPIODS | μ/δ | DAMGO, ME, | ↓AC-Gi/o | ↓ | NG; SO | ↓ | NG; SO |
| | | DPDPE (naloxone) | | | | | |
| | κ | U69593 (norBNI) | ↓AC-Gi/o | ↑ | NR | | |
| TACHYKININ | NK 1/2/3 | Sub P. ASMSP | ↑PLCβ ↑PLA2 | ˜ | NG | ↓ | −20 mV. NG |
| | | (CP99.994) | ↑AC | | | | |

B. Metabolism of phosphatidyl-inositol 4,5-bisphosphate and diacylglycerol

Phosphatidyl-inositol 4,5-bisphosphate (4,5-PIP₂) and diacylglycerol (DAG) are second messengers that are involved in a complex set of signaling cascades, which regulate a broad array of cellular responses, including survival, activation, differentiation, and proliferation.

The metabolism of 4,5-PIP₂ and DAG are regulated in part by lipid kinases, including phosphatidylinositol 3-kinases (PI3 Kinases), phosphatidylinositol 4-kinases (PI4 kinases) and phosphatidylinositol-4-P 5 kinases (PI5 kinases). In the classical pathway, phosphatidylinositol (PI) is phosphorylated at the 4′-OH position by PI4 kinases to form PI-4-P; then PI-4-P is phosphorylated at the 5-position by PI5 kinases to form PI 4,5-P₂ (Anderson et al, 1999, J. Biol. Chem. 274: 9907-9910). However, PI5 kinases are also capable of converting PI-5-P to PI 4,5-P₂ (Anderson et al, supra).

PI3 Kinases catalyse the addition of phosphate to the 3′-OH position of the inositol ring of inositol lipids generating phosphatidyl inositol monophosphate, diphosphate and triphosphate (Whitman et al, 1988, Nature 332: 644-646; Stephens et al, 1989, Biochem. J. 259: 267-276; Stephens et al., 1991, Nature 351: 33-39). PI3 kinases can phosphorylate 4,5-PIP₂ to form PI-3,4,5-P₃, which is dephosphrylated by PI-5 phosphatases to produce PI-3,4-P2, another second messenger. 4,5-PIP₂ is also a substrate for PLC. PLC hydrolyzes 4,5-PIP₂ to form DAG and Inositol-1,4,5-P₃. DAG is a substrate for the synthesis of triacylglycerol. It can be produced by dephosphorylation of 1,2-diacylglycerol phosphate (commonly identified as phosphatidic acid) by phosphatidic acid phosphatase. DAG can also be synthesized from monoacylglycerols.

It has been shown that recombinant RhoA stimulates PI5 kinase activity and RhoA can physical associate with PI5 kinase (Ren et al, 1996, Mol. Biol. Cell 7: 435-442). Rho-associated kinases (Rho-kinases) are serine/threonine kinases that act as Rho effectors. Overexpression of wild type Rho-kinase and the constitutively active catalytic domain of Rho-kinase, Rho-kinase-CAT stimulates both the PI5 kinase activity and 4,5-PIP₂ levels. The increase in PI5 kinase activity and 4,5-PIP₂ levels by wild type Rho-kinase is prevented by coexpression of C3 transferase, which acts to inactivate Rho (Weernink et al, 2000, J. Biol. Chem. 275: 10168-10174). These results suggest that 4,5-PIP₂ metabolism is regulated by a Rho-dependent pathway.

### SUMMARY OF THE INVENTION

The present invention is based on the surprising discovery that activation of HCN channel gating is modulated by second messengers, 4,5-PIP₂ and DAG. The applicants have found that 4,5-PIP₂ and DAG are positive modulators of HCN channel gating. The applicants also found that genistein, an inhibitor of tyrosine kinase, can either inhibit or facilitate HCN channel activation under different conditions. The present invention provides methods for modulating the activation of HCN channels, and for the treatment and prevention of disorders associated with abnormality in HCN channel functions. The present invention also provides methods of screening for compounds that modulate the activation of HCN channels. The present invention further encompasses compounds that modulate the activation of HCN channels as well as pharmaceutical compositions comprising such compounds.

In one aspect, the present invention provides a method of facilitating HCN channel activation in a cell comprising contacting a cell that expresses a HCN channel with an effective amount of a compound that increases the level of 4,5-PIP₂ in the cell.

In a further aspect, the present invention provides a method of inhibiting HCN channel activation in a cell comprising contacting a cell that expresses a HCN channel with an effective amount of a compound that decreases the level of 4,5-PIP₂ in the cell.

In another aspect, the present invention provides a method of screening for a compound that facilitates the activation of HCN channels comprising contacting a cell with a compound and determining whether the level of 4,5-PIP₂ is increased in the presence of the compound as compared to the level of 4,5-PIP₂ in the absence of the compound.

In another aspect, the present invention provides a method of screening for a compound that inhibits the activation of HCN channels comprising contacting a cell with a compound and determining whether the level of 4,5-PIP₂ is decreased in the presence of the compound as compared to the level of 4,5-PIP₂ in the absence of the compound.

In another aspect, the present invention provides a method of treating or preventing a disease or condition in an individual in which there is an undesirable reduction of the activity of HCN channels comprising administering to such individual a pharmaceutically effective amount of a compound that increases the level of 4,5-PIP₂ in the individual.

In another aspect, the present invention provides a method of treating or preventing a disease or condition in an individual in which there is an undesirable elevation of the activity of HCN channels comprising administering to such individual a pharmaceutically effective amount of a compound that decreases the level of 4,5-PIP₂ in the individual.

In a further aspect, the present invention provides a method of facilitating HCN channel activation in a cell comprising contacting a cell that expresses a HCN channel with an effective amount of a compound that increases the level of DAG in the cell.

In a further aspect, the present invention provides a method of inhibiting HCN channel activation in a cell comprising contacting a cell that expresses a HCN channel with an effective amount of a compound that decreases the level of DAG in the cell.

In another aspect, the present invention provides a method of screening for a compound that facilitates the activation of HCN channels comprising contacting a cell with the compound and determining whether the level of DAG is increased in the presence of the compound as compared to the level of DAG in the absence of the compound.

In another aspect, the present invention provides a method of screening for a compound that inhibits the activation of HCN channels comprising contacting a cell with a compound and determining whether the level of DAG is decreased in the presence of the compound as compared to the level of DAG in the absence of the compound.

In another aspect, the present invention provides a method of treating or preventing a disease or condition in an individual in which there is an undesirable reduction of the activity of HCN channels comprising administering to such individual a pharmaceutically effective amount of a compound that increases the level of DAG in the individual.

In another aspect, the present invention provides a method of treating or preventing a disease or condition in an individual in which there is an undesirable elevation of the activity of HCN channels comprising administering to such individual a pharmaceutically effective amount of a compound that decreases the level of DAG in the individual.

In a further aspect, the present invention provides a method of facilitating HCN channel activation in a cell comprising contacting a cell that expresses a HCN channel with an effective amount of a compound that stimulates the activity of a C1-binding protein that mediates the activation of a HCN channel by DAG.

In a further aspect, the present invention provides a method of inhibiting HCN channel activation in a cell comprising contacting a cell that expresses a HCN channel with an effective amount of a compound that inhibits the activity of a C1-binding protein that mediates the activation of a HCN channel by DAG.

In another aspect, the present invention provides a method of treating or preventing a disease or condition in an individual in which there is an undesirable reduction of the activity of HCN channels comprising administering to such individual a pharmaceutically effective amount of a compound that stimulates the activity of C1-binding protein that mediates the activation of a HCN channel by DAG in the individual.

In another aspect, the present invention provides a method of treating or preventing a disease or condition in an individual in which there is an undesirable elevation of the activity of HCN channels comprising administering to such individual a pharmaceutically effective amount of a compound that inhibits the activity of C1-binding protein that mediates the activation of a HCN channel by DAG in the individual.

In a further aspect, the present invention provides a method of facilitating HCN channel activation in a cell comprising contacting a cell that expresses a HCN channel with an effective amount of a first compound that increases the level of 4,5-PIP₂ in the cell, and a second compound that increases the level of DAG and/or stimulates the activity of a C1-binding protein that mediates the activation of a HCN channel by DAG in the cell.

In a further aspect, the present invention provides a method of inhibiting HCN channel activation in a cell comprising contacting a cell that expresses a HCN channel with an effective amount of a first compound that decreases the level of 4,5-PIP₂ in the cell, and a second compound that decreases the level of DAG and/or inhibits the activity of a C1-binding protein that mediates the activation of a HCN channel by DAG in the cell.

In another aspect, the present invention provides a method of treating or preventing a disease or condition in an individual in which there is an undesirable reduction of the activity of HCN channels comprising administering to such individual a pharmaceutically effective amount of a first compound that increases the level of 4,5-PIP₂ in the individual, and a second compound that increases the level of DAG and/or stimulates the activity of a C1-binding protein that mediates the activation of a HCN channel by DAG in the individual.

In another aspect, the present invention provides a method of treating or preventing a disease or condition in an individual in which there is an undesirable elevation of the activity of HCN channels comprising administering to such individual a pharmaceutically effective amount of a first compound that decreases the level of 4,5-PIP₂ in the individual, and a second compound that decreases the level of DAG and/or inhibits the activity of a C1-binding protein that mediates the activation of a HCN channel by DAG in the individual.

In another aspect, the present invention provides a method of screening for a compound that facilitates the activation of HCN channels comprising contacting a cell with a compound and determining whether the levels of 4,5-PIP₂ and DAG are increased in the presence of the compound as compared to the levels of 4,5-PIP₂ and DAG in the absence of the compound.

In another aspect, the present invention provides a method of screening for a compound that inhibits the activation of HCN channels comprising contacting a cell with a compound and determining whether the levels of 4,5-PIP₂ and DAG are decreased in the presence of the compound as compared to the levels of 4,5-PIP₂ and DAG in the absence of the compound.

In a further aspect, the present invention provides a method of inhibiting HCN channel activation in a cell comprising contacting a cell that expresses a HCN channel with an effective amount of a compound that inhibits the facilitation of HCN channel activation by 4,5-PIP₂ and/or DAG.

In another aspect, the present invention provides a method of treating or preventing a disease or condition in an individual in which there is an undesirable elevation of the activity of HCN channels comprising administering to such individual a pharmaceutically effective amount of a compound in the individual, wherein the compound inhibits the facilitation of HCN channel activation by 4,5-PIP₂ and/or DAG.

In another aspect, the present invention provides a method of screening for a compound that modulates the activation of HCN channels comprising contacting a cell that expresses a HCN channel with a test compound and a suitable amount of 4,5-PIP₂ and measuring the gating activity of the HCN channel.

In one aspect, the present invention provides a method of facilitating HCN channel activation in a cell by contacting a cell that expresses a HCN channel with a compound that inhibits the activity of a receptor tyrosine kinase, in the absence of cAMP or under a condition wherein the binding of cAMP to the CNBD domain of HCN channel is blocked.

In another aspect, the present invention provides a method of selectively inhibiting HCN2 channel activation in a cell comprising contacting a cell that expresses both a HCN1 channel and a HCN2 channel with a compound that inhibits the activity of a receptor tyrosine kinase in the presence of cAMP.

In a further aspect, the present invention provides a method of facilitating HCN1 channel activation but inhibiting HCN2 channel activation in a cell comprising contacting a cell that expresses both a HCN1 channel and a HCN2 channel with a first compound that selectively blocks the binding of cAMP to the CNBD domain of HCN1 channel and a second compound that inhibits the activity of a receptor tyrosine kinase.

In another aspect, the present invention provides compounds that facilitate or inhibit HCN channel activation and pharmaceutical compositions comprising such compounds.

Additional aspects of the present invention will be apparent in view of the description which follows.

### BRIEF DESCRIPTION OF THE FIGURES

FIG. 1. Schematic representation of the 910 amino acid mouse HCN1 ion channel subunit.
CARTOON: is scaled so that lineal distance of each segment is proportional to the number of residues in that element in HCN1.
STRUCTURAL ELEMENTS: WHITE and GREY boxes represent probable helices forming the transmembrane core of the channel. SIX VERTICAL WHITE boxes represent S1-S6 based upon homology to K⁺ channels. ANGLED GREY BOX and subsequent GYG sequence indicate the location of the pore helix and selectivity filter, respectively. The blue and red colored elements in the C-terminus represent the CNBD and the C-linker (the element that physically and energetically couples the CNBD to channel gating) as defined by the HCN2 crystal structure. In the CNBD, lines represent the strands of the beta roll and intervening disordered stretches. Helices and strands that contact cAMP are indicated by close apposition of those CNBD elements with the purine ring (B) and ribose phosphate (P-R) of the schematic nucleotide.
BLACK BOXES: leucine zipper (LZ) and PDZ domain.
CRITICAL ARGININE (indicated by red box and arrow): Forms an ionic bond with the cyclized phosphate of the CNBD and which, when mutated to glutamate renders the HCN channels insensitive to cAMP but does not alter basal gating or permeation properties.
CRITICAL HISTIDINE (indicated by blue box and arrow): Inhibits HCN gating when protonated. The channel is rendered insensitive to changes in internal pH when this residue is mutated to an arginine or glutamine.
SITES OF TRUNCATION (indicated by green dashed lines): Used to map second messenger coupling motifs. Deletions do not alter basal gating or permeation properties.

FIG. 2. I_{H} modulation: role in oscillatory activity and generation of persistent current.
A. I_{H} channels activate upon hyperpolarization and carry an inward current (BOTTOM). At more negative voltages (shown above), I_{H} activation is faster and more complete.
B. I_{H} facilitation or inhibition results in a larger or smaller current (BOTTOM) at a given voltage (TOP).
C. Steady-state activation curves in control (middle black line) and upon facilitation (right hand blue dashed line, +ΔV1/2) or inhibition (left hand red dashed line, −ΔV1/2). GREY shaded area indicates the current envelope active at a resting membrane potential of −70 mV.
D. Current clamp recording of a mouse ventrobasal thalamic relay cell. Current injection mimicking the persistent current upon strong facilitation of I_{H}, switches the cell from a bursting to a tonic firing mode. This transition is considered a cellular correlate of the unconscious/conscious transition.
E. View of bursting indicated in D. Smoothed blue line: schematic representation of change in burst frequency upon moderate facilitation of I_{H}.

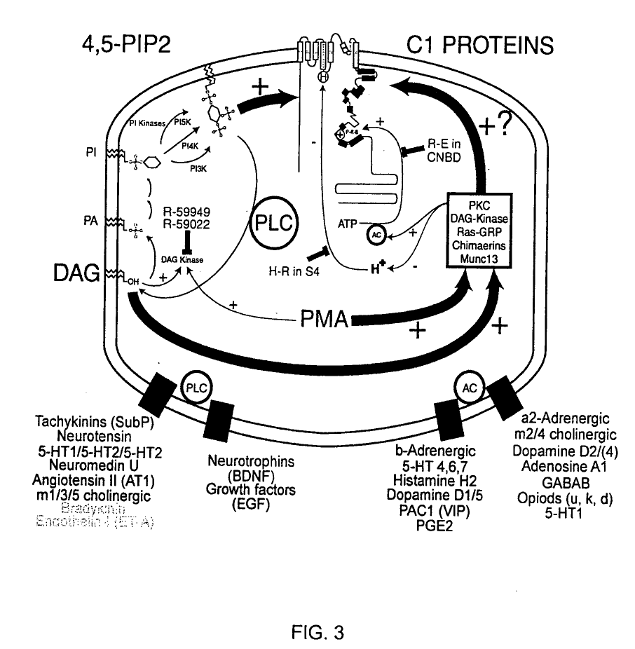

FIG. 3. Receptor and second messenger systems coupling to I_{H} channel activation.
Second messengers known to couple to expressed HCN channels are cAMP and H⁺'s. Ca is thought to couple to I_{H} channel activity through Ca-sensitive adenylate cyclase. Here, we describe 4,5-PIP₂ and DAG as new and previously undescribed modulators of HCN pacemaker channels (THICK BACK ARROWS).
GATING RESPONSE of native I_{H} channels to phospholipase C (PLC)/tyrosine kinase coupled receptors (LEFT) is indicated by the color of the receptor name: RED, inhibitory; BLUE facilitatory; GREEN: bimodal. Adenylate cyclase (AC) coupled receptors are shown on the RIGHT in BLACK
BLUE lines with plus signs and RED lines with minus signs show pathways of facilitation and inhibition, respectively.
THICK BLUE LINES: Mechanisms of HCN facilitation by 4,5-PIP₂ and DAG/4βPMA revealed by our preliminary studies.
THIN BLUE and THIN RED lines: Mechanisms here shown to not account for DAG/4βPMA regulation of HCN channels.
RED T SHAPES: Diagnostic inhibitory interventions. YELLOW OVAL: An unknown intermediate—possibly arachidonic acid.
THIN BLACK lines: Simplified version of the PLC and DAG-kinase mediated phosphoinositide/DAG cycle.

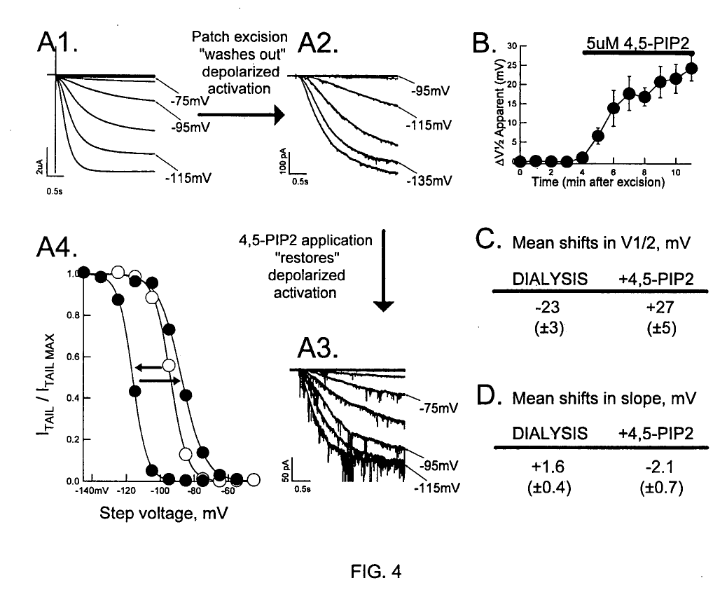

FIG. 4. “Rundown” activation gating of HCN2-R591E can be restored by 4,5-PIP₂.
A1-3. Records show HCN-R591E activation in 2MEV-clarnp (A1); in IOP-clamp after rundown has developed (A2); after 4,5-PIP₂ “rescue” of depolarized gating (A3). A4. Activation curves for the recordings shown in A1-3 (color coded accordingly) show hyperpolarization on patch dialysis (BLACK to RED) and 4,5-PIP₂ “recovery” (RED to BLUE).
B. Plot of the parameter “ΔV1/2 apparent” shows the time course of 4,5-PIP₂ rescue of depolarized gating.
C. Mean shifts in V1/2 suggest 4,5-PIP₂ contributes ˜20 mV to the intact cell gating of HCN2-R591E.
D. 4,5-PIP₂ restores the more shallow gating slope of HCN2-R591E observed in intact cells.

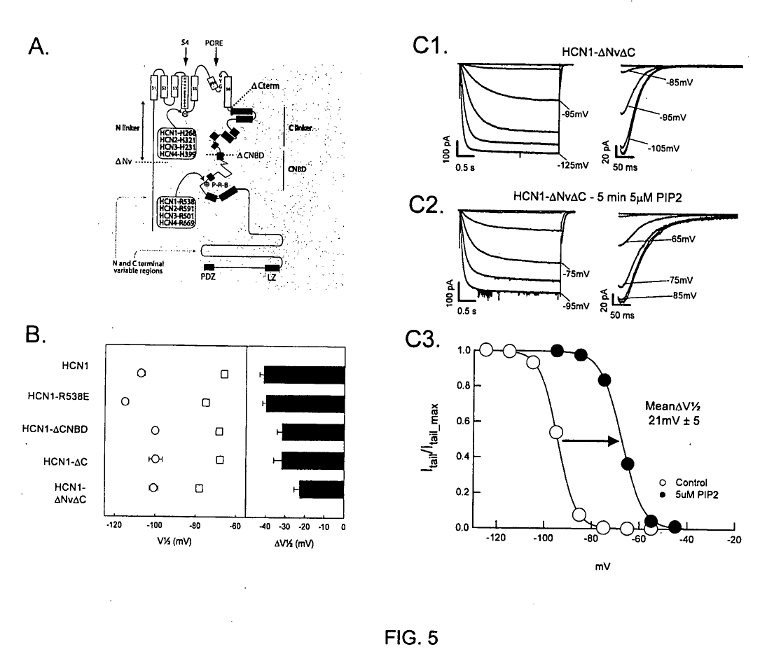

FIG. 5. Deletion analysis reveals that “rundown” and “4,5-PIP₂ reactivation” both map to the conserved core of HCN channels.
A. Schematic representation of an HCN subunit showing locations of deletion boundaries.
B. The hyperpolarizing shift in activation gating consequent upon loss of cellular factors is substantially retained in the N and C-terminally truncated channel, HCN1-ΔNvΔC. Left panel shows V1/2 of constructs determined in intact cells (□, 2MEV-clamp) and following dialysis dependent “rundown” in excised IOP-clamp (∘). Right panel shows the inhibitory shift in the V1/2 associated with patch excision.
C. Application of 5 uM 4,5-PIP₂ to a patch containing “rundown” HCN1-ΔNvΔC channels redepolarizes gating of this minimal channel. C1 shows currents recorded following “rundown” while C2 are records from the same patch following 5 min application of 5 uM 4,5-PIP₂. Inspection of the tail currents shows the depolarization of gating occurs without significant alteration of the tail current amplitude. C3 Activation curves constructed from the records in C1 and C2 and mean data from 6 such records reveals application of 4,5-PIP₂ (blue data) can “restore” HCN gating to the depolarized level observed in intact cells.

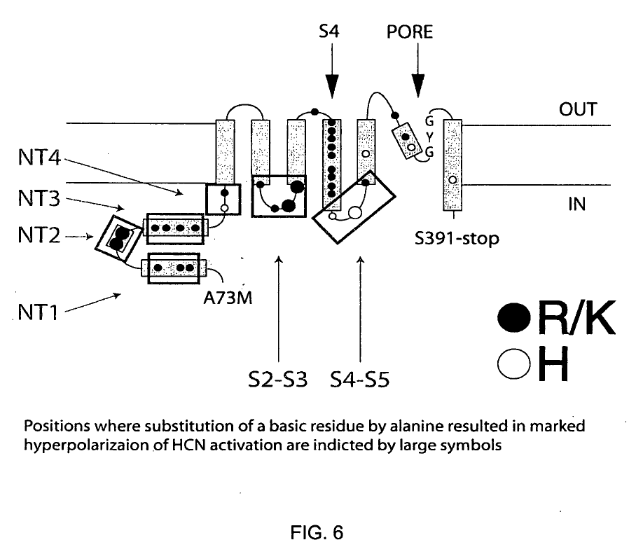

FIG. 6. Positions of basic residues in the minimal 4,5-PIP₂ responsive channel HCN1-ΔNvΔC.
Schematic representation of HCN1-ΔNvΔC showing location of basic residues and those mutated in our initial alanine replacement mutagenesis. Secondary structure prediction of the conserved N-linker domain shows that the two basic residues in the NT2 cassette are likely to be arranged as a helical stripe at the end of a “finger” formed from sequences including the residues located in NT1 and NT3.
It is hypothesized that anionic lipids such as 4,5-PIP₂ can form chemical, presumably ionic, bonds with specific residues in the channel. Such bonds will facilitate channel opening by specifically stabilizing the open state with respect to the closed state. This model predicts that the effects of the lipids will be mediated by a specific and identifiable subset of amino acids and that these residues will be located in cytoplasmically exposed parts of the channel protein.

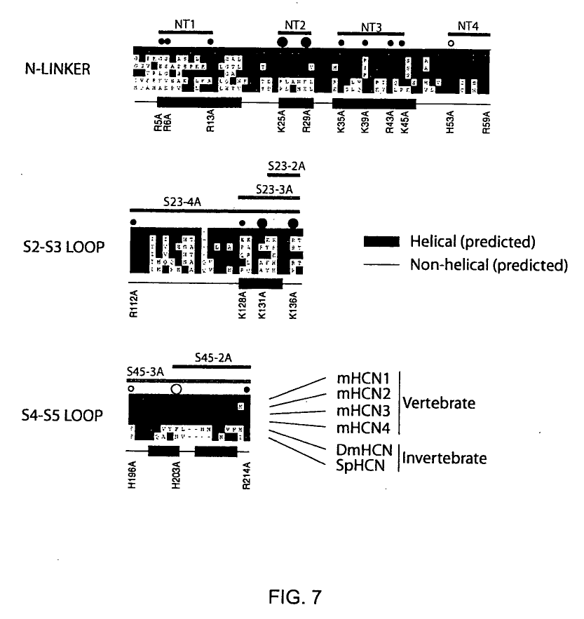

FIG. 7. Sequence alignments of the intracellular N-linker, S2-S3 AND S4-S5 loops of the minimal 4,5-PIP₂ responsive channel, HCN1-ΔNVΔC TO sequences from related HCN channels.
Alignment of the mHCN1 N-linker, S2-S3 and S4-S5 loops to homologous sequences from mHCN2-4 and the invertebrate HCN channels from Drosophila and Sea urchin. Predicted secondary structure of the mHCN1 sequences is shown under alignments.

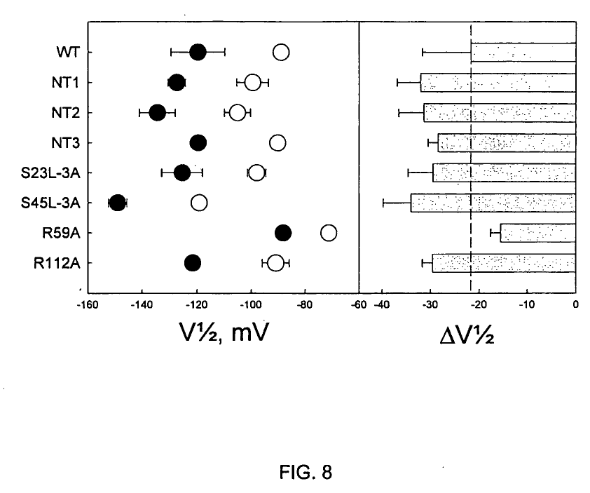

FIG. 8. Inside-out patch clamp analysis reveals that elimination of presumptive cytoplasmically exposed basic residues does not alter the sensitivity of HCN1-ΔNVΔC gating to changes in the surface charge potential.
THE LEFT PANEL reports the V1/2 for activation of HCN1-ΔNvΔC constructs wherein the indicated basic residues were mutated to alanine. OPEN CIRCLES show the V1/2 when the “intracellular” solution was devoid of polyvalent cations (Mg²⁺ or polylysine) that can act to screen the negative charges of anionic membrane lipids. CLOSED CIRCLES show the V1/2 when the “intracellular” solution was replaced with one that contained 1 mM free Mg²⁺ and polylysine. In all cases, channels gated at more negative potentials when the surface charge was shielded. Note that before these measurements were made, the patches were perfused with a solution that will promote depletion of the 4,5-PIP₂ so the surface charge effect under investigation here is that contributed by stable anionic lipids.
THE RIGTH PANEL reports the difference in the V1/2 in the presence and absence of surface charge shielding of stable anionic lipids. Importantly, this difference was not significant for any construct showing that the mutation had not significantly altered the ability of the voltage sensor to “sense” changes in surface charge. The BLUE dashed line shows the position of the mean wild type response.

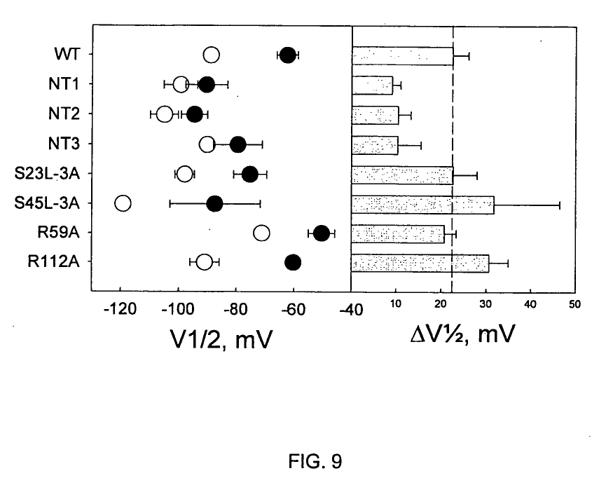

FIG. 9. Inside-out patch clamp analysis reveals that elimination of presumptive cytoplasmically exposed basic residues in the amino terminal Nv region (in cassettes NT1, NT2 AND NT3) greatly weakens coupling of the channels to 4,5-PIP₂— an effect not seen with any of the other presumptive cytoplasmically exposed basic residues suggesting the n-linker may be a site of interaction with anionic lipids including 4,5-PIP₂.
THE LEFT PANEL reports the V1/2 for activation of HCN1-ΔNvΔC constructs wherein the indicated basic residues were mutated to alanine. OPEN CIRCLES show the V1/2 when the “intracellular” solution was devoid of Mg²⁺—a condition necessary to protect subsequently applied 4,5-PIP₂ from that action of membrane associated lipases. Note that, preceeding the experiment, endogenous 4,5-PIP₂ was depleted by perfusion with a high Mg solution. CLOSED CIRCLES show the V1/2 when the “intracellular” solution was supplemented with 5 uM 4,5-PIP₂.
THE RIGHT PANEL reports the difference in the V1/2 in the presence and absence of 4,5-PIP₂. Importantly, the response of constructs NT1 and NT2 were significantly different from control (P<0.05) and that of NT3 approached significance even with this initial small sample number (P˜0.07). In contrast, R59A (P˜0.19; NT4 did not express); S23-3A (P˜1); R112A (P˜0.19; S23-4A did not express well enough to be measured), S45-3A (P˜0.3) were not different from wild type. The BLUE dashed line shows the position of the mean wild type response to 5 uM 4,5-PIP₂.

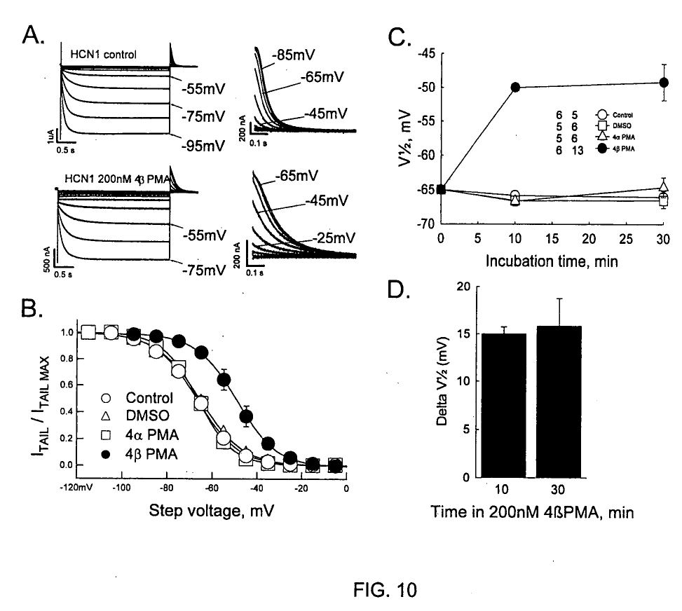

FIG. 10. 4βPMA but not 4αPMA facilitates gating of HCN1
A. 2MEV-clamp current families (LEFT) and expanded view of the tail currents (RIGHT) from cells expressing HCN1. Currents were recorded in the absence (TOP) or following 30 min incubation in the presence (BOTTOM) of 200 nM 4βPMA. Cells were held at −30 mV. Immediately prior to the 3s activation step a cell is stepped to +20 mV for 1s to deactivate channels open at the holding potential and then stepped to the test potential (voltages indicated by arrows attached to select traces).
B. Mean steady state activation curves following 30 min incubation in the absence or presence of 200 nM 4βPMA, 4αPMA, DMSO vehicle alone.
C. Plot of the V1/2 of activation versus time of incubation in the indicated drug. The V1/2 in 4βPMA at both 10 and 30 min was significantly different (P<0.0005) from all of the control groups (n for each time is indicated sequentially next to appropriate legend) but the three control conditions were not different from each other.
D. 4βPMA elicited ΔV1/2=(V1/2+4βPMA)−(V1/2 control).

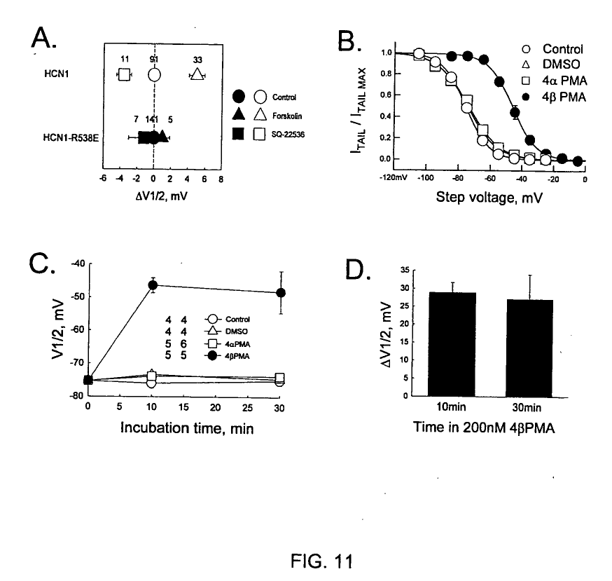

FIG. 11. The 4βPMA facilitation of HCN1 is not mediated by cAMP.
A. Plots of the V1/2 shows activation of HCN1 is facilitated by the adenylate cyclase activator forskolin (10 μM, 30 min) but not the inactive analogue dideoxy-forskolin (not shown) and inhibited by SQ-22536 (an adenylate cyclase inhibitor; 300 μM, 3 hr). Both differences were significant, P<0.0005. Gating of HCN1-R538E is not altered by either cyclase activation or inhibition (P>0.5 that each drug regime is from the same population as control).
B. Steady-state activation curves following 30 min incubation in the absence or presence of 200 nM 4βPMA, 200 nM 4αPMA, DMSO vehicle or untreated control.
C. Plot of the V1/2 of activation versus time of incubation in the indicated drug. The V1/2 in 4βPMA at both 10 and 30 min was significantly different (P<0.005) from all of the control groups (n for each time is indicated sequentially next to appropriate legend) but the three control conditions were not different from each other.
D. 4βPMA elicited ΔV1/2=(V1/2+4βPMA)−(V1/2 control).

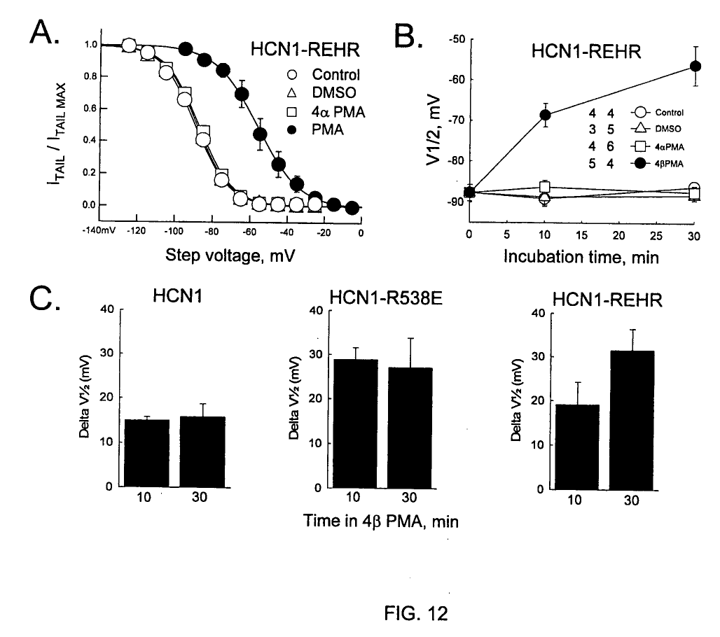

FIG. 12. Elimination of the proton binding site at Histidine 268 does not abolish 4βPMA facilitation of HCN1.
A. Steady-state activation curves of HCN1-REHR following 30 min incubation in the absence or presence of 200 nM 4βPMA, 4αPMA, DMSO vehicle alone.
B. Plot of the V1/2 of activation versus time of incubation in the indicated condition. The V1/2 in 4βPMA at both 10 and 30 min was significantly different (P<0.005) from all of the control groups (n for each time is indicated sequentially next to appropriate legend) but the three control conditions were not different from each other.
C. Summary plot showing the 4βPMA elicited ΔV1/2 for wtHCN1, HCN1-R538E and HCN1-REHR. In each case the data are (V1/2+4βPMA)−(V1/2 control).

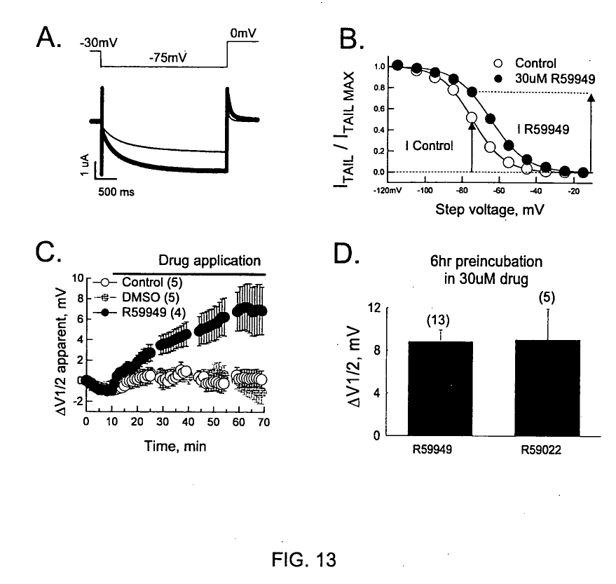

FIG. 13. Blockade of diacylglycerol kinase facilitates HCN1-R538E activation.
A. Activation and deactivation of the CNBD-disabled channel HCN1-R538F BEFORE (BLACK TRACE) and AFTER (THICK BLUE TRACE) perfusion of the cell with 30 uM R59949 for 60 min. Activation is to −75 mV, the V1/2 prior to drug treatment. A larger current is elicited after drug reflecting channel facilitation.
B. Activation curves before (∘) and after (●) 60 min perfusion with R59949 (cell is that shown in A).
C. Plot of “ΔV1/2 apparent” shows onset of response to R59949, DMSO vehicle compared to untreated controls. The V1/2 in R59949 60 min was significantly different from zero time and from 60 min DMSO and untreated control groups (P<0.005). The n for each condition is next to appropriate legend.
D. Shift in V1/2 for drug treated cells versus control cells. The V1/2 in each drug was different (P<0.0005) from paired controls. Numbers of drug treated cells is shown above bars.

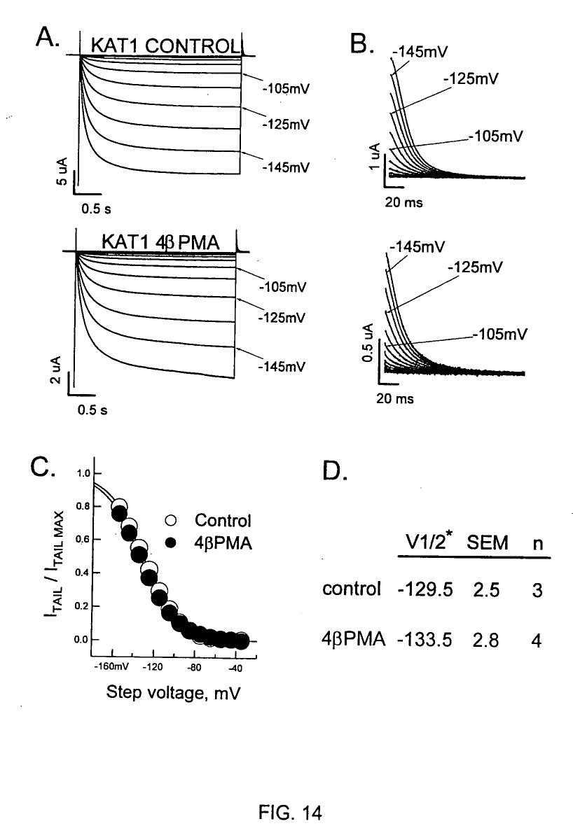

FIG. 14. Steady state activation of KAT1 (a plant HCN channel) is not altered by 200 nM 4βPMA.
A, B. Activation and deactivation records of KAT1 in the absence and presence of 200 nM 4βPMA.
C. Activation curves for recordings shown in A and B. Although the fit of the Boltzmann equation is compromised by the failure of KAT1 channel activation to saturate in the accessible voltage range, the data indicate that activation was not significantly altered by 4βPMA.
D. Mean V1/2* of KAT 1 activation in the presence and absence of 4βPMA. *denotes the poor constraint on the fit as noted above.

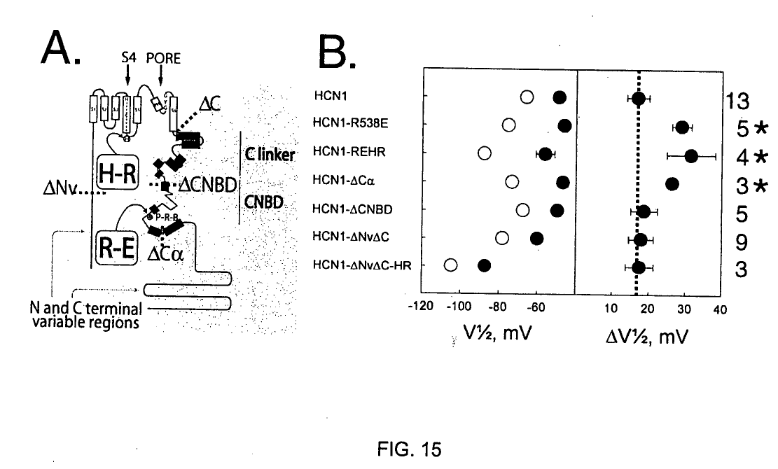

FIG. 15. Deletion mapping of domains required for the 4βPMA response in HCN1 and 2.
A. Schematic diagram showing deletions (green dashed lines) and point mutations (H-R and R-E) used in constructs reported in B. Green shaded area shows the extent of deletion when the ΔNv and ΔC deletions are combined.
B. LEFT panel shows V1/2 of indicated construct following incubation in the absence (OPEN CIRCLES) or presence of 200 nM 4βPMA (FILLED BLUE CIRCLES). In each case the populations were different (P<0.005 or better). RIGHT panel shows extent of facilitation—difference between control and 4βPMA V1/2—for each clone. Where indicated by an asterisk, the facilitation for that clone was significantly different (P<0.05 or better) from the facilitation reported for wt HCN1 (top entry).

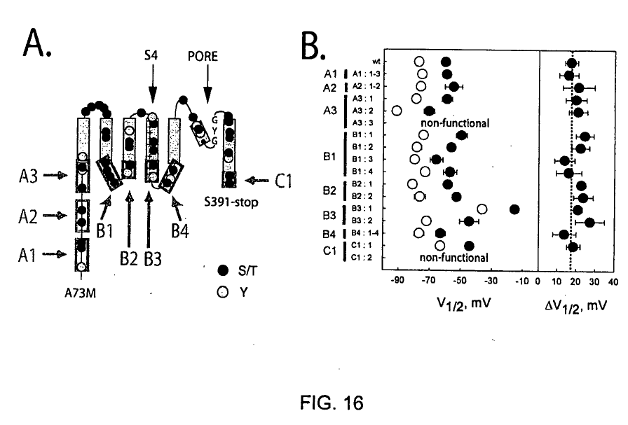

FIG. 16. Elimination of phosphorylatable residues does not blunt 4βPMA facilitation of HCN gating.
A. To probe the role of channel phosphorylation either by PKC directly or other kinases downstream of 4βPMA, we mutated serines in consensus PKC sites (2 serines in box A2) and all other serines, threonines and tyrosines. Mutations were constructed in the background of HCN1-ΔNvΔC as shown and were S/T to N and Y to F.
B. LEFT: V1/2 following 30 min incubation in the presence (FILLED BLUE CIRCLES) or absence (OPEN CIRCLES) of 200 nM 4βPMA. V1/2 of all clones was significantly depolarized relative to paired controls (P<0.05 or better, n was >4 for all groups). RIGHT: Plot of ΔV1/2 for each construct. In no case was facilitation decreased compared to the parental channel, HCN1-ΔNvΔC (Top entry and BLUE DASHED REFERENCE LINE). Two constructs failed to generate functional channels.

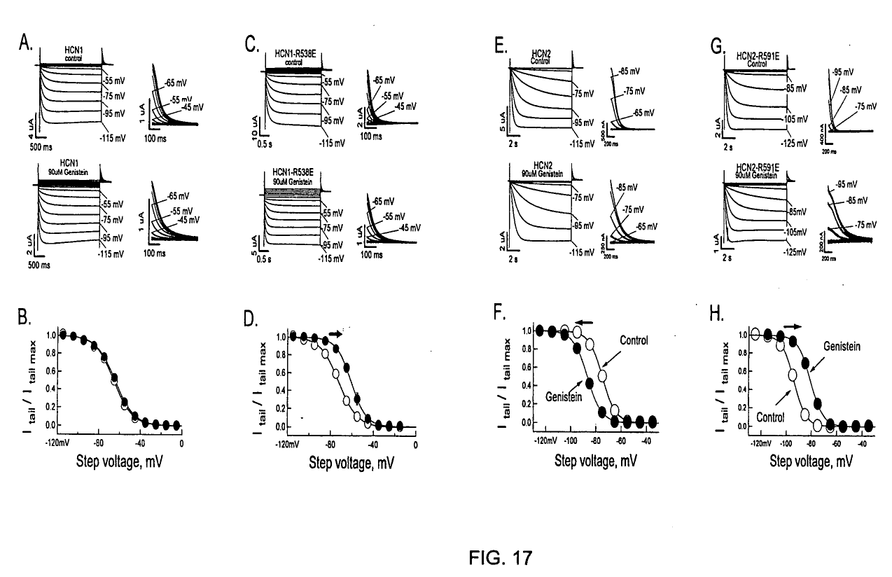

FIG. 17. Nature and efficacy of modulation of HCN activation gating by the PTK inhibitor genistein is determined by nucleotide-occupancy of the channels cyclic nucleotide binding pocket.
A,C,E,G. Show the gating of wild type and CNBD-disabled HCN channels in the absence (top) and presence (bottom) of the PTK inhibitor, genistein (A: HCN1; C:HCN1-R538E; E: HCN2; G: HCN2-R591E).
B,D,F,H. Activation curves for each construct in the absence and presence of genistein reveal that the weak facilitation of HCN1 by genistein (B) converts to a robust facilitation when the CNBD is disabled (D) (Right shifts shown in BLUE). In HCN2, the response to genistein converts from an inhibition (F) (left shift shown in RED) to facilitation (Right shift shown in BLUE) when the CNBD is disabled (H).

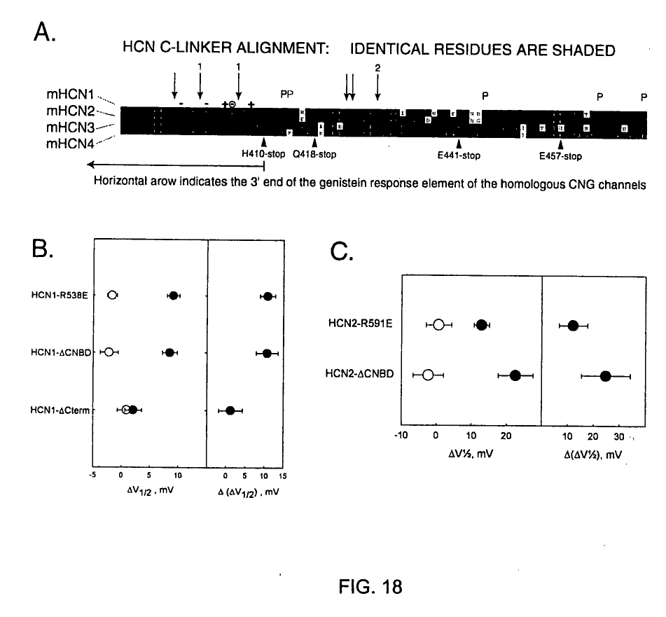

FIG. 18. Domain deletion analysis reveals that the HCN C-linker but not the CNBD is required for regulation of gating by the PTK inhibitor, genistein.
A. Alignment of the C-linker domains of mHCN1-4. ARROWS: Tyrosine residues. NUMBERS: High probability tyrosine phosphorylation sites identified by NetPhos2 (1) and Procite (2). + and − signs indicate position in the C-linker of the related CNG channels that, when mutated to histidine, can coordinate Ni and facilitate or inhibit channel gating, respectively.
B. LEFT: Change in V1/2 (V1/2 Final−V1/2 Initial) in control (∘) and genistein treated cells (●) (90 μM for 30 min). RIGHT: Plot of Δ(ΔV1/2) shows genistein facilitation is independent of the CNBD (facilitation of HCN1-ΔCNBD and full length HCN1-R538E are the same) but requires the C-linker, as its deletion renders HCN1-ΔCterm insensitive to genistein.
C. Genistein facilitation of cAMP insensitive HCN2 constructs is also independent of the CNBD.

### DETAILED DESCRIPTION OF THE INVENTION

The present invention relates to the modulation of HCN channel gating by second messengers, 4,5-PIP₂ and DAG. The present invention provides methods for modulating the activation of HCN channels, and for the treatment and prevention of disorders associated with abnormality in HCN channel functions. The present invention also provides methods of screening for compounds that modulate the activation of HCN channels. The present invention further encompasses compounds that facilitate or inhibit the activation of HCN channels as well as pharmaceutical compositions comprising such compounds.

The invention is based in part on the surprising discovery by the applicants that 4,5-PIP₂ and DAG are positive modulators of HCN channel gating. 4,5-PIP₂ directly facilitates HCN channel gating while DAG facilitation of HCN channel gating is mediated by a C1-binding protein. The region of the HCN channel protein required for responding to the modulators is the core region of the HCN channel. Furthermore, the applicants have found that basic amino acids on the cytoplasmic region of HCN channels are involved in the interaction with 4,5-PIP₂. In particular, evidences showed that two basic amino acids clusters, located between the N-terminal variable region and the transmembrane region of the HCN channels, for example, NT1 and NT2 of mHCN1 as shown in FIG. 6 and FIG. 7, may directly interact with 4,5-PIP₂.

4,5-PIP₂ and DAG are substrate and product of PLC, respectively. The activation of PLCs would effect a yin-yang form of regulation of HCN channel gating since both the substrates and products of the PLCs could positively couple to channel activation (FIG. 3). On the one hand, activation of PLCs would inhibit HCN channel activation by dis-facilitation through lowering of 4,5-PIP₂. On the other hand, activation of PLCs would facilitate HCN channels activation if the appropriate signal cascades were positioned close to the channels such that they could respond to increase in IP3/Ca and/or DAG acting via C1-binding proteins. Thus, the net effect of PLC activation on HCN channel activation depends on the balancing effect of the decrease in 4,5-PIP₂ and increase in DAG. PLCs play an important role in modulating the activation of HCN channels.

The present invention is also based in part on the discovery that a receptor tyrosine kinase (PTK) inhibitor, genistein, can alter HCN channel activation. However, the effect of genistein treatment on HCN channel activation is dependent on cAMP binding to the CNBD domain of the channel. Also, genistein treatment has different effect on HCN1 channels and HCN2 channels. In particular, genistein facilitation of channel gating is weak in the wild-type HCN1 channel but robust in the HCN1-R538E construct wherein the CNBD domain is disabled. Gating of HCN2 channel is inhibited by genistein in the wild-type HCN2 channel but facilitated in the HCN2-R591E construct in which the CNBD domain is disabled. Together, these data demonstrate that PTKs may be involved in HCN regulation consistent with a role of PTK modulation as a basis for trophic receptor mediated changes in HCN function.

Although the response of a full-length HCN channel to genistein is determined by the cAMP occupancy of the CNBD, the ability of the channel to sense genistein depends on the presence of only the coupling motif (the C-linker) but not upon the CNBD itself.

The present invention relates to methods of modulating HCN channel gating in cells or individuals based on one or more of the mechanisms described above. The present invention further relates to screening assays to identify compounds that modulate HCN channel gating by one or more of the mechanisms described above. The present invention also encompasses compounds that modulate HCN channel gating by one or more of the mechanisms described above and pharmaceutical compositions comprising such compounds.

A. Definitions

As used herein, the terms “treat,” “treating” and “treatment” refer to a method of alleviating or eliminating a disease, disorder or condition, its attendant symptoms, and/or the cause of the disease, disorder or condition itself.

As used herein, the terms “prevent,” “preventing” and “prevention” refer to a method of delaying or precluding the onset of a disease, disorder or condition and/or its attendant symptoms, barring a subject from acquiring a disease, disorder or condition or reducing a subject's risk of acquiring a disease, disorder or condition.

As used herein, the term “effective amount” refers to the amount of a compound that will elicit the biological or medical response of a cell, tissue, system, animal or human that is being sought by the researcher, veterinarian, medical doctor or other clinician. “Pharmaceutically effective amount” may further include the amount of a compound that, when administered, is sufficient to prevent development of, or alleviate to some extent, or eliminate, one or more of the symptoms of the disease, disorder or condition being treated.

As used herein, the term “individual” is defined herein to include animals such as mammals, including, but not limited to, primates (e.g., humans), cows, sheep, goats, horses, dogs, cats, rabbits, rats, mice and the like. In preferred embodiments, the individual is a human.

Other terms will be evident as used in the following description.

B. Second Messenger Modulation of HCN Channel Activation

The applicants have shown that 4,5-PIP₂ could directly facilitate the activation of HCN channels. Accordingly, increasing the level of 4,5-PIP₂ within a cell would facilitate the activation of HCN channels of the cell and decreasing the level of 4,5-PIP₂ within a cell would inhibit the activation of HCN channels. It is also believed that other phosphoinositides would have the same regulatory effect on HCN channels as 4,5-PIP₂.

The applicants have also found that inhibition of the activity of DAG-kinase facilitates the activation of HCN channels, suggesting that DAG is also a positive modulator of HCN channel gating. The effect of DAG-facilitation of HCN channel activation is stereoselectively mimicked by 4-βphorbol-1 2myristate-13-acetate (4βPMA), a C1-binding protein ligand, but not the C1-inactive 4αPMA analogue. Those results indicate that DAG may facilitate the activation of HCN channels via a C1-binding protein, for example, without limitation, PKC, PKD, Ras-GRP, Chimaerins and Munc13. Therefore, modulating the level of DAG and the activity of C1-binding protein would also regulate the activation of HCN channels.

In one aspect, the present invention provides a method of facilitating HCN channel activation in a cell comprising contacting a cell that expresses a HCN channel with an effective amount of a compound that increases the level of 4,5-PIP₂ in the cell. In one embodiment, the compound is exogenous 4,5-PIP₂. 4,5-PIP₂ is commercially available from Calbiochem (San Diego, Calif.).

In another embodiment, the compound increases the level of 4,5-PIP₂ in the cell by stimulating the production of endogenous 4,5-PIP₂ by the cell. 4,5-PIP₂ is synthesized from PI by the successive actions of PI4 kinases and PI5 kinases. Activators of PI4 kinases and PI5 kinases as well as other proteins involved in the synthesis pathway of 4,5-PIP₂ would stimulate the production of endogenous 4,5-PIP₂. In one embodiment, the compound useful to the method of the invention is an activator of PI4 kinases and/or PI5 kinases. In a preferred embodiment, the compound useful to the method of the invention is RhoA, or Rho-kinase, or Rho-kinase-CAT. It has been shown that overexpression of RhoA, Rho-kinase or Rho-kinase-CAT in a cell stimulates PI5 kinase activity and increases 4,5-PIP₂ levels (Weernink et al, 2000, J. Biol. Chem. 275: 10168-10174). RhoA, Rho-kinase or Rho-kinase-CAT can be obtained by recombinant expression or protein purification using standard methods well known in the art (see, for example, Weernink et al, supra). Standard methods for producing recombinant proteins will be described below.

In another embodiment, the compound increases the level of 4,5-PIP₂ in the cell by inhibiting the depletion of endogenous 4,5-PIP₂ in the cell. 4,5-PIP₂ is a substrate for PI3 kinases. Inhibition of the activity of PI3 kinases would increase the level of 4,5-PIP₂ in a cell. Thus, in one embodiment, the compound that stimulates the level of 4,5-PIP₂ in a cell is an inhibitor of PI3 kinases. In a preferred embodiment, the PI3 kinase inhibitor is wortmannin, or derivatives or analogues of wortmarinin that retain the ability to inhibit PI3 kinases. Examples of wortmannin derivatives and analogues are disclosed in Wiesinger, D. et al, 1974, Experientia 30:135-136; Closse, A. et al, 1981, J. Med. Chem. 24:1465-1471; and Baggiolini, M. et al, 1987, Exp. Cell Res. 169:408-418. In another embodiment, the PI3 inhibitor is bioflavenoid quercetin, or derivatives or analogues of quercetin that retain the ability to inhibit PI3 kinases. Examples of quercetin derivatives and analogues are disclosed in Vlahos, C. J. et al, 1994, J. Biol. Chem. 269:5241-5284. A preferred quercetin derivative that inhibits PI3 kinase activity is LY294002 (Vlahos et al, supra).

In a further aspect, the present invention provides a method of inhibiting HCN channel activation in a cell comprising contacting a cell that expresses a HCN channel with an effective amount of a compound that decreases the level of 4,5-PIP₂ in the cell. In one embodiment, the compound is capable of directly binding to 4,5-PIP₂. In a preferred embodiment, the compound is a monoclonal antibody that specifically binds to 4,5-PIP₂. A highly specific mAb against 4,5-PIP₂, KT10, is commercially available (Assay Designs, Inc., Ann Arbor, Mich.; Fukami et al, 1988, Proc. Natl. Acad. Sci. USA 85: 9057-9061; Matuoka et al, 1988, Science 239: 640-643). In another embodiment, the compound is a phosphatase that dephosphorylates 4,5-PIP₂. Such phosphatase should dephosphorylate 4,5-PIP₂ but not dephosphorylate a precursor of DAG to produce DAG. In one embodiment, the phosphatase is a 5-phosphatase or phosphoinositide phosphatase.

In another embodiment, the compound decreases the level of 4,5-PIP₂ in the cell by inhibiting the endogenous production of 4,5-PIP₂ by the cell. In one embodiment, the compound is an inhibitor of PI4 kinases and/or PI5 kinases. In a preferred embodiment, the compound is adenosine, an inhibitor of PI4 kinase activity. In a more preferred embodiment, the compound is arachidonic acid, which inhibits the activity of both PI4 kinases and PI5 kinases. Both adenosine and arachidonic acid are commercially available from Sigma-Aldrich Corp. (St. Louis, Mo.).

In another embodiment, the compound decreases the level of 4,5-PIP₂ in the cell by stimulating the depletion of endogenous 4,5-PIP₂ in the cell. In one embodiment, the compound is an activator of PI3 kinases.

In another aspect, the present invention provides a method of screening for a compound that facilitates the activation of HCN channels comprising contacting a cell with a compound and determining whether the level of 4,5-PIP₂ is increased in the presence of the compound as compared to the level of 4,5-PIP₂ in the absence of the compound. In another aspect, the present invention provides a method of screening for a compound that inhibits the activation of HCN channels comprising contacting a cell with the compound and determining whether the level of 4,5-PIP₂ is decreased in the presence of the compound as compared to the level of 4,5-PIP₂ in the absence of the compound. The level of 4,5-PIP₂ in a cell can be measured by any suitable method known in the art. Examples of such methods are high pressure liquid chromatography and thin layer chromatography (see, for example, Okada, T. et al. (1994) J. Biol. Chem. 269:3563-3567). The present invention also encompasses the compounds that are identified through the screening methods described herein, as capable of modulating HCN channel gating through modulating the levels of 4,5-PIP₂ in the cell.

The “cell that expresses a HCN channel” may express endogenous and/or exogenous HCN channel proteins. In one embodiment, the cell expresses abundant endogenous HCN channels such as a cardiac cell or a neuronal cell. In another embodiment, the cell is a recombinant cell that expresses exogenous HCN channel proteins. Methods of producing a cell that expresses a HCN channel have been described (Santoro et al, 1997, Proc. Natl. Acad. Sci. USA 94: 14815-14820; Santoro et al, 1998, Cell 93:717-729; Ludwig et al, 1998, Nature 393: 587-591; U.S. Pat. No. 6,703,485).

A recombinant cell expressing an exogenous protein can be produced using standard molecular biology methods well known in the art (see, for example, Sambrook et al. (“Molecular Cloning, A Laboratory Manual.” Cold Spring Harbor Press, Cold Spring Harbor, N.Y. 1989). Briefly, a recombinant cell expressing a protein is produced by introducing a nucleotide sequence encoding a protein into an expression vector, transfecting or transforming such expression vector into a host cell, culturing the host cell under suitable condition so that the host cell expresses the protein. If recombinant protein is desired, standard methods well known in the art could be applied to purify the protein (see, for example, Sambrook et al, supra).

The nucleic acid may be synthesized using commercially available oligonucleotide synthesis instrumentation (Gait, M. J. Ed., Oligonucleotide Synthesis, IRL Press, Oxford, 1984). Preferablly, the nucleic acid encoding a HCN protein is produced by the recombinant DNA technology. To obtain the nucleic acid by recombinant technology, a cDNA library is prepared from mRNA isolated from cells expressing the HCN protein; an oligonucleotide probe having a partial sequence of the desired HCN gene is synthesized using commercially available oligonucleotide synthesis instrumentation; the probe is used to screen the cDNA library for clones that hybridize to the probe, or the probe is used to amplify the HCN gene from the cDNA library by a polymerase chain reaction (PCR). The cDNA clone or clones so obtained are excised by suitable restriction enzymes, and ligated into a suitable expression vector for protein expression.

Both eukaryotic and procaryotic expression systems can be used to express the HCN proteins. Preferably, genes encoding HCN protein are expressed in eucaryotic host cell cultures derived from multicellular organisms (See, e.g., Tissue Cultures, Academic Press, Cruz and Patterson, Eds, (1973)). Useful host cell lines include Xenopus oocytes, COS cells, VERO and HeLa cells, Chinese hamster ovary (CHO) cells, and insect cells such as SF9 cells. Expression vectors for such cells ordinarily include promoters and control sequences compatible with mammalian cells such as, for example, the commonly used early and late promoters from baculovirus, vaccinia virus, Simian Virus 40 (SV40) (Fiers et al., 1973, Nature 273:113), or other viral promoters such as those derived from polyoma, Adenovirus 2, bovine papilloma virus, or avian sarcoma viruses. The controllable promoter, hMTII (Karin et al., 1982, Nature 299:797-802) may also be used.

Prokaryotes most frequently are represented by various strains of E. coli; however, other microbial species and strains may also be used. Commonly used prokaryotic control sequences include promoters for transcription initiation, optionally with an operator, along with ribosome binding site sequences, including such commonly used promoters as the β-lactamase (penicillinase) and lactose (lac) promoter systems (Chang et al., 1977, Nature 198:1056) and the tryptophan (trp) promoter system (Goeddel et al., 1980, Nucl Acids Res 8:4057) and the λ derived P_{L} promoter and N-gene ribosome binding site (Shimatake et al., 1981, Nature 292:128).

Depending on the host cell used, transformation is carried out using standard techniques appropriate to such cells. The treatment employing calcium chloride, as described by Cohen, 1972, Proc. Natl. Acad. Sci. USA 69:2110 or by Sambrook et al. (supra), can be used for prokaryotes or other cells which contain substantial cell wall barriers. For mammalian cells without such cell walls, the calcium phosphate precipitation method of Graham and van der Eb, 1978, Virology 54:546, optionally as modified by Wigler et al., 1979, Cell 16:777-785, or by Chen and Okayama, supra, can be used. Transformations into yeast can be carried out according to the method of Van Solingen et al., 1977, J. Bact. 130:946, or of Hsiao et al., 1979, Proc. Natl. Acad. Sci. USA 76:3829.

Other representative transfection methods include viral transfection, DEAE-dextran mediated transfection techniques, lysozyme fusion or erythrocyte fusion, scraping, direct uptake, osmotic or sucrose shock, direct microinjection, indirect microinjection such as via erythrocyte-mediated techniques, and/or by subjecting host cells to electric currents. The above list of transfection techniques is not considered to be exhaustive, as other procedures for introducing genetic information into cells may be developed.

In another aspect, the present invention provides a method of treating or preventing a disease or condition in an individual in which there is an undesirable reduction of the activity of HCN channels comprising administering to such individual a pharmaceutically effective amount of a compound that increases the level of 4,5-PIP₂ in the individual. In one embodiment, the compound is exogenous 4,5-PIP₂. In another embodiment, the compound stimulates the production of endogenous 4,5-PIP₂ in the individual. Preferably, such stimulation is specific to the target cells or tissues of the individual. In another embodiment, the compound inhibits the depletion of endogenous 4,5-PIP₂ in the individual. Preferably, such inhibition is specific to the target cells or tissues of the individual.

In another aspect, the present invention provides a method of treating or preventing a disease or condition in an individual in which there is an undesirable elevation of the activity of HCN channels comprising administering to such individual a pharmaceutically effective amount of a compound that decreases the level of 4,5-PIP₂ in the individual. In one embodiment, the compound inhibits the endogenous production of 4,5-PIP₂ in the individual. Preferably, such inhibition is specific to the target cells or tissues of the individual. In another embodiment, the compound stimulates the depletion of endogenous 4,5-PIP₂ in the individual. Preferably, such stimulation is specific to the target cells or tissues of the individual.

Abnormality in the function of HCN channels is suggested to be associated with major pathologies such as neuropathic pain, epilepsy and changes associated with hypertrophied or heart failure. It has also been reported that genes encoding proteins that are involved in the synthesis and dephosphorylation of 4,5-PIP₂ are located in overlapping regions of the genome with loci mapped in schizophrenia, suggesting that 4,5-PIP₂ metabolism may be linked to schizophrenia (Stopkova et al, 2003, Am. J. Med. Genet. B. Neuropsychiatr. Genet. 123: 50-58). HCN channel dysfunction has also been implicated in motor learning dysfunction and sick sinus syndrome. In one embodiment, the treatment methods of the invention may be used to treat an individual suffering from neuropathic pain, epilepsy, hypertrophied, heart failure, schizophrenia, motor learning dysfunction and sick sinus syndrome. Samples may be taken from such individuals to measure the level of activation of the HCN channels in such individuals and determine whether there is an elevation or reduction in HCN channel activation from the normal values. Methods for measuring HCN channel activation are described in the Examples and are well known in the art.

In a further aspect, the present invention provides a method of facilitating HCN channel activation in a cell comprising contacting a cell that expresses a HCN channel with an effective amount of a compound that increases the level of DAG in the cell. In one embodiment, the compound is exogenous DAG. In another embodiment, the compound stimulates the production of endogenous DAG by the cell. In another embodiment, the compound inhibits the depletion of endogenous DAG in the cell. In one embodiment, the compound is a DAG-Kinase inhibitor. In a preferred embodiment, the compound is R59022 or R59949. In a more preferred embodiment, the compound is R59949.

In a further aspect, the present invention provides a method of inhibiting HCN channel activation in a cell comprising contacting a cell that expresses an HCN channel with an effective amount of a compound that decreases the level of DAG in the cell. In one embodiment, the compound is capable of depleting the free DAG in the cell. In a preferred embodiment, the compound is an agent that directly binds to DAG. In another embodiment, the compound inhibits the production of endogenous DAG by the cell. In another embodiment, the compound stimulates the depletion of endogenous DAG in the cell. In one embodiment, the compound is an activator of DAG-kinase.

In another aspect, the present invention provides a method of screening for a compound that facilitates the activation of HCN channels comprising contacting a cell with the compound and determining whether the level of DAG is increased in the presence of the compound as compared to the level of DAG in the absence of the compound. In another aspect, the present invention provides a method of screening for a compound that inhibits the activation of HCN channels comprising contacting a cell with the compound and determining whether the level of DAG is decreased in the presence of the compound as compared to the level of DAG in the absence of the compound. The level of DAG in a cell can be measured by thin layer chromatography or any other suitable method known in the art.

In another aspect, the present invention provides a method of treating or preventing a disease or condition in an individual in which there is an undesirable reduction of the activity of HCN channels comprising administering to such individual a pharmaceutically effective amount of a compound that increases the level of DAG in the individual. In one embodiment, the compound is exogenous DAG. In another embodiment, the compound stimulates the production of endogenous DAG in the individual. Preferably, such stimulation is specific to the target cells or tissues of the individual. In another embodiment, the compound inhibits the depletion of endogenous DAG in the individual. Preferably, such inhibition is specific to the target cells or tissues of the individual. In another embodiment, the compound is a DAG-kinase inhibitor. In another embodiment, the compound is R59022 or R59949. In a more preferred embodiment, the compound is R59949.

In another aspect, the present invention provides a method of treating or preventing a disease or condition in an individual in which there is an undesirable elevation of the activity of HCN channels comprising administering to such individual a pharmaceutically effective amount of a compound that decreases the level of DAG in the individual. In one embodiment, the compound inhibits the production of endogenous DAG in the individual. Preferably, such inhibition is specific to the target cells or tissues of the individual. In another embodiment, the compound stimulates the depletion of endogenous DAG in the individual. Preferably, such stimulation is specific to the target cells or tissues of the individual. In one embodiment, the compound is a DAG-kinase activator.

In a further aspect, the present invention provides a method of facilitating HCN channel activation in a cell comprising contacting the cell with an effective amount of a compound that stimulates the activity of a C1-binding protein that mediates the activation of a HCN channel by DAG. In one embodiment, the C1-binding protein is a PKC, PKD, Ras-GRP, Chimaerins, or Munc13. In a preferred embodiment, the C1-binding protein is a PKC. In another embodiment, the compound is 4βPMA.

In a further aspect, the present invention provides a method of inhibiting HCN channel activation in a cell comprising contacting the cell with an effective amount of a compound that inhibits the activity of a C1-binding protein that mediates the activation of a HCN channel by DAG. In one embodiment, the C1-binding protein is a PKC, PKD, Ras-GRP, Chimaerins, or Munc13. In a preferred embodiment, the C1-binding protein is a PKC.

In another aspect, the present invention provides a method of treating or preventing a disease or condition in an individual in which there is an undesirable reduction of the activity of HCN channels comprising administering to such individual a pharmaceutically effective amount of a compound that stimulates the activity of C1-binding protein that mediates the activation of a HCN channel by DAG in the individual. In one embodiment, the C1-binding protein is a PKC, PKD, Ras-GRP, Chimaerins, or Munc13. In a preferred embodiment, the C1-binding protein is a PKC. In another embodiment, the compound is 4βPMA.

In another aspect, the present invention provides a method of treating or preventing a disease or condition in an individual in which there is an undesirable elevation of the activity of HCN channels comprising administering to such individual a pharmaceutically effective amount of a compound that inhibits the activity of C1-binding protein that mediates the activation of a HCN channel by DAG in the individual. In one embodiment, the C1-binding protein is a PKC, PKD, Ras-GRP, Chimaerins, or Munc13. In a preferred embodiment, the C1-binding protein is a PKC.

In another aspect of the invention, the level of DAG and the activity of C1-binding protein are both changed to facilitate or inhibit the activation of HCN channels.

The 4,5-PIP₂- and DAG-mediated mechanisms could be used together to effectively modulate HCN channel gating. They could also be used in combination with other mechanisms of modulating HCN channel gating.

In a further aspect, the present invention provides a method of facilitating HCN channel activation in a cell comprising contacting the cell with an effective amount of a first compound that increases the level of 4,5-PIP₂ in the cell, and a second compound that increases the level of DAG and/or stimulate the activity of a C1-binding protein that mediates the activation of a HCN channel by DAG in the cell. In a preferred embodiment, the first compound is exogenous 4,5-PIP₂ and the second compound is exogenous DAG. In another preferred embodiment, the first compound is exogenous 4,5-PIP₂ and the second compound is R59022, R59949 or 4BPMA. The first and second compound may be the same compound.

In a further aspect, the present invention provides a method of inhibiting HCN channel activation in a cell comprising contacting the cell with an effective amount of a first compound that decreases the level of 4,5-PIP₂ in the cell, and a second compound that decreases the level of DAG and/or inhibits the activity of a C1-binding protein that mediates the activation of a HCN channel by DAG in the cell.

In another aspect, the present invention provides a method of treating or preventing a disease or condition in an individual in which there is an undesirable reduction of the activity of HCN channels comprising administering to such individual a pharmaceutically effective amount of a first compound that increases the level of 4,5-PIP₂ in the individual, and a second compound that increases the level of DAG and/or stimulates the activity of a C1-binding protein that mediates the activation of a HCN channel by DAG in the individual. In a preferred embodiment, the first compound is exogenous 4,5-PIP₂ and the second compound is exogenous DAG. In another embodiment, the first compound is exogenous 4,5-PIP₂ and the second compound is R59022, R59949, or 4βPMA. The first and second compound may be the same compound.

In another aspect, the present invention provides a method of treating or preventing a disease or condition in an individual in which there is an undesirable elevation of the activity of HCN channels comprising administering to such individual a pharmaceutically effective amount of a first compound that decreases the level of 4,5-PIP₂ in the individual, and a second compound that decreases the level of DAG and/or inhibits the activity of a C1-binding protein that mediates the activation of a HCN channel by DAG in the individual. The first compound and the second compound may be the same compound.

In another aspect, the present invention provides a method of screening for a compound that facilitates the activation of HCN channels comprising contacting a cell with the compound and determining whether the levels of 4,5-PIP₂ and DAG are increased in the presence of the compound as compared to the levels of 4,5-PIP₂ and DAG in the absence of the compound. In another aspect, the present invention provides a method of screening for a compound that inhibits the activation of HCN channels comprising contacting a cell with the compound and determining whether the levels of 4,5-PIP₂ and DAG are decreased in the presence of the compound as compared to the levels of 4,5-PIP₂ and DAG in the absence of the compound.

The applicants have revealed that the core region of the HCN channels is required for the 4,5-PIP₂- and DAG-dependent facilitation of channel activation. As illustrated in FIG. 5A, the core region in mHCN1 channel is the portion of the HCN channel without the green shaded N and C-terminal regions. In general, the core region refers to the portion of the HCN Channel that lacks the N and C terminal variable regions. The mHCN1 channel core region spans from amino acid residue 73 to amino acid residue 390. The core region is highly conserved among HCN isoforms from the same or different species. It was suggested that 4,5-PIP₂ interacts with the basic amino acid residues in the cytoplasmic region of the HCN channel, in particular, the cytoplasmic portion of the core region of HCN channels. The applicants have shown that mutation of the basic amino acid residues to alanine in the NT1 NT2; and NT3 cluster of mHCN1 (see FIG. 7) inhibits 4,5-PIP₂ mediated facilitation of HCN channel activation. In particular, mutation in NT1 and NT2 clusters showed stronger inhibitory effect. As shown in FIG. 7, NT1 has three point mutations (R5A, R6A and R13A), NT2 has two (K25A and R29A), NT3 has four (K35A, K39A, R43A and K45A). FIG. 7 also shows that different HCN isoforms share high sequence homology in the cytoplasmic region covering NT1-3. As used herein, the term “NT1,” “NT2,” and “NT3” include homologous regions of different HCN isoforms.

In another aspect, the present invention provides a method of inhibiting HCN channel activation in a cell comprising contacting a cell that expresses a HCN channel with an effective amount of a compound that inhibits the interaction between 4,5-PIP₂ and a HCN channel. In one embodiment, the compound inhibits the interaction between 4,5-PIP₂ and the core region of the HCN channel. In another embodiment, the compound inhibits the interaction between 4,5-PIP₂ and the portion of the core region that is exposed to the cytoplasmic side of the cell. In another embodiment, the compound inhibits the interaction between 4,5-PIP₂ and the NT1, NT2, or NT3 regions of the HCN channel. In a preferred embodiment, the compound inhibits the interaction between 4,5-PIP₂ and the NT1 or NT2 region of the HCN channel. In another preferred embodiment, the compound inhibits the interaction between 4,5-PIP₂ and one or more basic amino acid residues in the NT1 and NT2 regions. In another embodiment, the compound inhibits the interaction between 4,5-PIP₂ and one or more basic amino acid residues in the cytoplasmic region of the HCN channel. In another embodiment, the compound is an agent that chemically modifies the basic amino acids of the HCN channel. In another embodiment, the compound is capable of binding to the HCN channel via non-covalent bonds. In a preferred embodiment, the compound is a monoclonal or polyclonal antibody.

In another aspect, the present invention provides a method of inhibiting HCN channel activation in a cell comprising contacting a cell that expresses a HCN channel with an effective amount of a compound that inhibits the interaction between phosphoinositides and the HCN channel. The phosphoinositides may be phosphoinositide monophosphate, phosphoinositide diphosphate, or phosphoinositide triphosphate. In a preferred embodiment, the phosphoinositides are phosphoinositide diphosphates, preferably, the phosphoinositide diphosphates are 4,5-PIP₂.

In another aspect, the present invention provides a method of facilitating HCN channel activation in a cell comprising contacting a cell that expresses a HCN channel with an effective amount of a compound that stimulates the interaction between phosphoinositides and the HCN channel. The phosphoinositides may be phosphoinositide monophosphate, phosphoinositide diphosphate, or phosphoinositide triphosphate. In a preferred embodiment, the phosphoinositides are phosphoinositide diphosphates, preferably, 4,5-PIP₂.

In a further aspect, the present invention provides a method of inhibiting HCN channel activation in a cell comprising contacting the cell with an effective amount of a compound that inhibits the facilitation of HCN channel activation by DAG. In a preferred embodiment, such compound is an antibody that binds to the core region of HCN channels.

In another aspect, the present invention provides a method of screening for a compound that modulates the activation of HCN channels comprising contacting a cell that expresses a HCN channel with a test compound and a suitable amount of 4,5-PIP₂ and measuring the gating activity of the HCN channel. If in the presence of the test compound, 4,5-PIP₂ mediated HCN channel activation is further facilitated, the test compound is an agonist of HCN channel activation. On the other hand, if in the presence of the test compound, 4,5-PIP₂ mediated HCN channel activation is inhibited or reduced, the test compound is an antagonist of HCN channel activation. Methods for measuring HCN channel activation are described in the Examples and are well known in the art. Agonists and antagonists of the HCN channel activation may be used as therapeutic or prophylactic agents to treat diseases associated with HCN channel dysfunction.

In another aspect, the present invention provides a method of treating or preventing a disease or condition in an individual in which there is an undesirable elevation of the activity of HCN channels comprising administering to such individual a pharmaceutically effective amount of a compound in the individual, wherein the compound inhibits the interaction between the HCN channel and 4,5-PIP₂. In one embodiment, such compound is an antibody that binds to the core region of HCN channels. In another embodiment, such compound is an antibody that binds to the NT1 and/or NT2 cluster of the HCN channels.

In another aspect, the present invention provides a method of treating or preventing a disease or condition in an individual in which there is an undesirable elevation of the activity of HCN channels comprising administering to such individual a pharmaceutically effective amount of a compound in the individual, wherein the compound inhibits the facilitation of HCN channel activation by DAG. In one embodiment, such compound is an antibody that binds to the core region of HCN channels.

Antibodies that specifically bind to the core region of HCN channels are prepared by immunizing suitable mammalian hosts according to known immunization protocols using peptides representing the core region of HCN channels or fragments thereof. The protein sequences of various HCN channel isoforms have been disclosed and the core region of the HCN channel identified (Santoro et al, 1997, Proc. Natl. Acad. Sci. USA 94: 14815-14820; Santoro et al, 1998, Cell 93:717-729; Ludwig et al, 1998, Nature 393: 587-591; U.S. Pat. No. 6,703,485).

Peptides representing the core region of HCN channels or fragments thereof can be produced using standard solid phase (or solution phase) peptide synthesis methods, as is known in the art (see, for example, Stewart et al., Solid Phase Peptide Synthesis, 2nd ed., Pierce Chem. Co., Rockford, Ill. (1984)). In addition, the peptides may be produced by recombinant DNA technology as well known in the art (see, for example, Sambrook et al, supra). The DNA encoding the core region may be synthesized using commercially available oligonucleotide synthesis instrumentation (Gait, M. J. Ed., Oligonucleotide Synthesis, IRL Press, Oxford, 1984) for production of the peptides using the recombinant DNA technology.

To enhance immunogenicity, the peptides are generally coupled to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co., Rockford, Ill., may be desirable to provide accessibility to the immunogen.

Administration of the immunogens is conducted generally by injection over a suitable time period and with use of suitable adjuvants, as is generally understood in the art. During the immunization schedule, titers of antibodies are taken to determine adequacy of antibody formation.

Various host animals can be immunized by injection with the peptides. Host animals include, without limitation, rabbits, mice, guinea pigs, and rats. Various adjuvants that can be used to increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin, and dinitrophenol. Potentially useful human adjuvants include BCG (bacille Calmette-Guerin) and Corynebacterium parvum.

While the polyclonal antisera produced in this way may be satisfactory for some applications, for pharmaceutical compositions, use of monoclonal antibody (mAb) preparations is preferred.

Monoclonal antibodies can be prepared using the desire peptides. Immortalized cell lines which secrete the desired mAbs may be prepared using standard hybridoma technology (see, for example, Kohler et al., Nature 256:495, 1975; Kohler et al., Eur. J. Immunol. 6:511. 1976; Kohler et al., Eur. J. Immunol. 6:292, 1976; Hammerling et al., Monoclonal Antibodies and T Cell Hybridomas, Elsevier, N.Y. 1981). The immortalized cell lines secreting the desired mAbs are screened by immunoassay using the desired antigen. When the appropriate immortalized cell culture secreting the desired mAb is identified, the cells can be cultured either in vitro or by intraperitoneal injection into animals wherein the mAbs are produced in the ascites fluid.

The desired mAbs are then recovered from the culture supernatant or from the ascites fluid. In addition to intact antibodies, fragments of the mAbs or of polyclonal antibodies which contain the antigen-binding portion can be used in the methods of the invention. Use of immunologically reactive antigen binding fragments, such as the Fab, Fab′, of F(ab′)₂ fragments, is often preferable, especially in a therapeutic context, as these fragments are generally less immunogenic than the whole immunoglobulin molecule. Such antibody fragments can be generated by known techniques. For example, F(ab′)₂ fragments can be produced by pepsin digestion of the antibody molecule, and Fab fragments can be generated by reducing the disulfide bridges of F(ab′)₂ fragments.

The present invention provides compounds that facilitate or inhibit HCN channel activation and pharmaceutical compositions comprising such compounds.

C. Receptor Tyrosine Kinase-Mediated Activation of HCN Channels

It was found that genistein, an inhibitor of receptor tyrosine kinases, has different effect on HCN1 channels and HCN2 channels, depending on the cAMP-occupancy on the CNBD domain of the channels. Upon cAMP occupancy of the CNBD domain, the kinase inhibitor weakly facilitates the activation of HCN1 channels but inhibits the activation of HCN2 channels. In the absence of cAMP occupancy of the CNBD domain, the kinase inhibitor strongly facilitates the activation of both the HCN1 channels and the HCN2 channels.

In one aspect, the present invention provides a method of facilitating HCN channel activation in a cell by contacting the cell with a compound that inhibits the activity of a receptor tyrosine kinase, in the absence of cAMP or under a condition wherein the binding of cAMP to the CNBD of HCN channel is blocked. In a preferred embodiment, the compound is genistein. In one embodiment, the binding of cAMP to the CNBD is blocked by antibodies against CNBD.

In another aspect, the present invention provides a method of selectively inhibiting HCN2 channel activation in a cell comprising contacting the cell with a compound that inhibits the activity of a receptor tyrosine kinase in the presence of cAMP. The term “selectively” means that the compound inhibits HCN2 channel activation but not the activation of other HCN isoforms such as HCN1 channel. In a preferred embodiment, the compound is genistein.

In a further aspect, the present invention provides a method of facilitating HCN1 channel activation but inhibiting HCN2 channel activation in a cell comprising contacting the cell with a first compound that blocks the binding of cAMP to the CNBD of HCN1 channel but does not block the binding of cAMP to the CNBD of HCN2 channel, and a second compound that inhibits the activity of a receptor tyrosine kinase. In the method, the compound selectively blocks the CNBD of HCN1 channel but would not block the CNBD of other HCN channel isoforms such as HCN1. In a preferred embodiment, the first compound is an antibody that specifically binds to the CNBD domain of HCN1 channel but not the CNBD domain of the HCN2 channel, the second compound is genistein. Antibodies binding to CNBD may be produced using standard methods well known in the art, as described above. The amino acid sequence of the CNBD of HCN1 and HCN2 channels have been identified (Santoro et al, 1997, Proc. Natl. Acad. Sci. USA 94: 14815-14820; Santoro et al, 1998, Cell 93:717-729; Ludwig et al, 1998, Nature 393: 587-591; U.S. Pat. No. 6,703,485).

In another aspect, the present invention provides a method of distinguishing the gating activity of HCN1 channel and HCN2 channel in a cell comprising contacting the cell with a compound that inhibits the activity of a receptor tyrosine kinase in the presence of cAMP, measuring the activation of the channel. If the channel activation is facilitated upon the addition of the compound, the channel is a HCN1 channel. If the channel activation is inhibited upon the addition of the compound, the channel is a HCN2 channel.

D. Pharmaceutical Formulations and Methods of Administration

Pharmaceutical compositions employed in the treatment methods of the invention may be prepared using standard methods well known in the art. The pharmaceutical compositions may comprise one or more compounds of the invention as active ingredients, and may further comprise one or more other pharmaceutically acceptable ingredients, including an excipient (a compound that provides a desirable property or activity to the composition, but other than or in addition to that of the active ingredient), a carrier, an adjuvant, a diluent, a vehicle, or the like.

The compounds of the invention may be modified by appropriate functionalities to enhance the desired biological properties. Such modifications are known in the art and include those, which increase the ability of the compounds to penetrate or being transported into a given biological system (e.g., circulatory system, lymphatic system), increase oral availability, increase solubility to allow administration by injection, alter metabolism of the compounds, and alter the rate of excretion of the compounds. In addition, the compounds of the invention may be altered to a pro-drug form such that the desired compounds are created in the body of an individual as the result of the action of metabolic or other biochemical processes on the pro-drug. Such pro-drug forms typically demonstrate little or no activity in in vitro assays. Some examples of pro-drug forms may include ketal, acetal, oxime, and hydrazone forms of compounds, which contain ketone or aldehyde groups. Other examples of pro-drug forms include the hemi-ketal, hemi-acetal, acyloxy ketal, acyloxy acetal, ketal, and acetal forms.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene polyoxypropylene block polymers, polyethylene glycol and wool fat.

The pharmaceutical compositions used in the methods of this invention may be administered by a variety of routes or modes. These include, but are not limited to, parenteral, oral, intratracheal, sublingual, pulmonary, topical, rectal, nasal, buccal, vaginal, or via an implanted reservoir. Implanted reservoirs may function by mechanical, osmotic, or other means. The term “parenteral”, as understood and used herein, includes intravenous, intracranial, intraperitoneal, paravertebral, periarticular, periostal, subcutaneous, intracutaneous, intra-arterial, intramuscular, intra articular, intrasynovial, intrasternal, intrathecal, and intralesional injection or infusion techniques. Such compositions are preferably formulated for parenteral administration, and most preferably for intravenous, intracranial, or intra-arterial administration. Generally, and particularly when administration is intravenous or intra-arterial, pharmaceutical compositions may be given as a bolus, as two or more doses separated in time, or as a constant or non-linear flow infusion.

The dosage to be administered, and the mode of administration will depend on a variety of factors including age, weight, sex, condition of the individual, and genetic factors, and will ultimately be decided by the attending physician or veterinarian. In general, dosage required for therapeutic efficacy will range from about 0.001 to 25.0 mg/kg of host body weight.

Details concerning dosages, dosage forms, modes of administration, composition and the like are further discussed in a standard pharmaceutical text, such as Remington's Pharmaceutical Sciences (1990), which is incorporated herein by reference.

The present invention is described in the following Examples, which are set forth to aid in the understanding of the invention, and should not be construed to limit in any way the scope of the invention as defined in the claims which follow thereafter.

### EXAMPLES

The following materials and methods were used to perform the experiments described in the Examples below:

Molecular Biology. Murine HCN1 and HCN2 were subcloned into the pGH19 and pGHE expression vector, respectively (Wainger et al., 2001, Nature 411: 805-810; Goulding et al., 1994, Nature 372: 369-374). Point mutation and truncation constructs were made by introducing premature stop codons and XbaI sites by PCR, except for HCN1-ΔCterm, for which an oligonucleotide linker containing a stop codon and an XbaI site was used. Fragments containing the mutations were subcloned into the parent channel using an endogenous PflMI site and the XbaI site. We sequenced regions generated by PCR or linker at least twice to verify the mutations. RNA was transcribed from NheI-linearized DNA or SphI-linearized DNA using T7 RNA polymerase (Message Machine kit; Ambion, Houston, Tex.) and injected into Xenopus oocytes prepared as previously described (Santoro et al., 1998).

Electrophysiology-General. Recordings were made in either IOP (excised cell-free inside out patch) or 2 MEV (two microelectrode voltage clamp) configurations from Xenopus oocytes 1-5 days after cRNA injection. In both configurations, the Ag—AgCl ground wire(s) were connected to the bath solution by 3M KCl-2% agar salt bridge electrodes placed downstream of, but close to, the oocyte. All recordings were obtained at room temperature (23-25° C.).

Electrophysiology-Excised cell-free inside-out patch clamp recordings. Data were acquired using an Axopatch 200B integrating patch clamp amplifier (Axon Instruments, USA), filtered at 1 kHz then digitized at 2 kHz using an ITC-18 interface (strutech), and recorded with Pulse software (Heka Electronics). The pipette solution contained (in mM): 107 KCl, 5 NaCl, 10 HEPES (free acid), and 2 MgCl₂, pH 7.4, with KOH, with 2 MgCl₂ replaced in some experiments with 1 MgCl₂ and 1 CaCl₂; the bath solution contained (in mM): 107 KCl, 5 NaCl, 10 HEPES (free acid), 1 MgCl₂, and 1 EGTA (free acid), pH 7.4, with KOH with, in some experiments, the MgCl₂ replaced with 1 EGTA. The hyperpolarizing shift in gating fully develops in 3 min after patch excision when the extracellular (pipette) solution contains Ca and Mg and the intracellular (bath) solution contains 1 MgCl₂ and 1 EGTA. When cAMP was applied, NaCl was replaced with equivalent amount of cAMP. In experiments where 4,5-PIP₂, kinase inhibitors or other compounds were included, stock solutions of the compounds dissolved in either DMSO or H₂O were added to the recording solutions to an appropriate final concentration. The volume of the compound stock solutions did not significantly alter concentrations of the other solutes. It was routinely tested and confirmed that addition of the solvents alone was without effect.

Electrophysiology-Two microelectrode voltage-clamp recordings. Data were acquired using a Warner Instruments (Hamden, Conn.) OC-725B amplifier, filtered at 1 kHz then digitized at 2 kHz using an ITC-18 interface and recorded with Pulse software (as described above). For most experiments, oocytes were bathed in a recording solution containing (in mM): 107 KCl, 5 NaCl, 10 HEPES (free acid), and 2 MgCl₂, pH 7.4, with NaOH. However, to screen for poorly expressing constructs a recording solution containing (in mM): 112 KCl, 10 HEPES (free acid), and 2 MgCl₂, pH 7.4, with KOH, was used to maximize the amplitude of the currents. Under these conditions, tail currents were recorded at the holding potential, −30 mV. The microelectrodes were filled with 3M KCl and had resistances of 0.1-0.5 MΩ (I passing) and 1-4 MΩ (V sensing). An active virtual ground was used to clamp the bath.

Electrophysiology—Whole Cell Patch Clamp Recordings. Data were acquired using an Axopatch 200B integrating patch clamp amplifier, filtered at 1 kHz then digitized at 2 kHz using an ITC-18 interface, and recorded with Pulse software. The pipette solution contained (in mM): 5 KCl, 130 NaCl, 1 CaCl₂, 1 MgCl₂, 10 Glucose, 10 HEPES (free acid), pH 7.4, with KOH; the bath solution contained (in mM): 120 KCl, 5 NaCl, 10 EGTA-K2, 1 CaCl₂, 1 MgCl₂, 10 HEPES (free acid), pH 7.4, with KOH.

Analysis of electrophysiological recordings. “Steady-state” activation curves were determined from the amplitude of tail currents observed after hyperpolarizing voltage steps on return to the indicated potential. For IOP-clamp experiments, the holding potential and the tail potential was −40 mV. For 2 MEV-clamp experiments, the oocytes were held at −30 mV and the tail amplitudes measured at 0 mV. To avert any systematic bias of the determination of the V_{1/2} in a particular drug and clone combination, at least 10 cells from at least 5 donor frogs were routinely measured with recordings made across a number of weeks.

Tail current amplitudes were measured by averaging the current during the plateau of the tail after allowing the voltage-clamp to settle and the uncompensated linear capacitance to decay and subtracting from this the baseline current recorded 3s later. Changing the durations or positions of these windows had no effect on activation curves. Current values were plotted versus the hyperpolarization step voltage and fitted with the Boltzmann equation:
I(V=A₁+A₂/{1+exp[(V−V_{1/2})/s]},
where A₁ is an offset caused by a nonzero holding current, A₂ is the maximal tail current amplitude, V is voltage during the hyperpolarizing test pulse in millivolts, and V_{1/2} is the activation midpoint voltage. For each experiment, the tail current data were fitted with the above equation. To average the activation data from different experiments, the tail current amplitudes, I(V), from each individual experiment were normalized by first subtracting the derived A₁ parameter and then dividing by A₂. The normalized data at each voltage were then averaged, and the averaged data were fitted by the Boltzmann equation (with A₁ set at 0 and A₂ set at 1). Custom analysis routines were written with Igor Pro (Wavemetrics Corp.). All data presented are means±s.e.m.

### Example 1

### 4,5-PIP₂ Rescues the Depolarized Gating of HCN2-R591E

An HCN2-R591E construct was expressed in Xenopus oocytes. 2 MEV-clamp recording was performed to measure the gating activity of the HCN2-R591 E channel in the intact Xenopus oocytes. IOP-clamp was performed to measure the gating activity of the HCN2-R591E channel under a “rundown” condition. IOP-clamp was also performed in the presence of 4,5-PIP₂ to detect the effect of 4,5-PIP₂ on the gating activity of the HCN2-R591E channel under a “rundown” condition. The results in FIG. 4 showed that 4,5-PIP₂ can restore the gating activity of HCN2-R591E channel under a “rundown” condition back to what it is in intact cells.

### Example 2

### The Core Region of HCN Channels are Required for Responding to 4,5-PIP₂

Wild type HCN1, and HCN1 mutants, HCN1-R538E (replacement of arginine at position 538 with glutamic acid), HCN1-ΔCα (deletion of the extreme C terminus and 25 amino acid C helix of HCN1 (Y565stop)), HCN1-ΔCNBD (deletion of the CNBD and extreme C terminus of HCN1 (V473stop)), HCN1-ΔNvΔCterm (ΔNv and ΔCterm double mutants: inclusion of an artificial start codon after removal of the preceding 5′ sequence for HCN1 (A73M), and deletion of the entire C terminus (C linker, CNBD and extreme C terminus) of HCN1 (S391stop)), were expressed in Xenopus oocytes. 2 MEV-clamp was performed to measure the gating activity of the wild type and mutant channels in intact cells. IOP-clamp was performed to measure the gating activity of the wild type and mutant channels under a “rundown” condition. The results in FIG. 5 shows that deletion or point mutation of the CNBD, N and C terminal variable regions did not disrupt the ability of the HCN channel to respond to 4,5-PIP₂. It indicates that the core region of the HCN channel is substantially but perhaps not exclusively involved in the interaction with 4,5-PIP₂.

### Example 3

### Elimination of Cytoplasmically Exposed Residues Does not Alter the Sensitivity of HCN1 Gating

The minimal 4,5-PIP₂ responsive mouse HCN1 channel mutant, HCN1-ΔNvΔCterm (amino acid positions 73 to 390 of mHCN1), were further mutated to produce the following constructs in which the basic amino acid residues Arginine, Lysine and Histidine are replaced with Alanine: NT1 (R5A, R6A, and R13A), NT2 (K25A, and R29A), NT3 (K35A, K39A, R43A, and K45A), NT4 (H53A, and R59A), S23-4A (R112A, K128A, K131A, and K136A), S23-3A (K128A, K131A, and K136A), S23-2A (K131A, and K136A), S45-3A (H196A, H203A, and R214A), and S45-2A (H203A, and R214A). See FIG. 7. All individual point mutations were also constructed. The mutants were expressed in Xenopus oocytes.

IOP clamp was performed with the mutant channels in the presence and absence of 1 mM free Mg²⁺ plus or minus polylysine in the bath solution. The results in FIG. 8 show that the HCN1-ΔNvΔCterm mutant and the other mutants with additional mutation in basic residues have similar sensitivity to changes in the surface charge potential.

IOP clamp was performed with the mutant channels in the presence and absence of 5 μM 4,5-PIP₂. The results in FIG. 9 show that mutants NT1, NT2 and NT3 have a reduced response to 4,5-PIP₂ facilitation of channel gating. It suggests that the basic amino acid residues R5, R6, R13, K25, R29, K35, K39, R43, and K45 of mHCN1-ΔNvΔCterm (also known as R77, R78, R85, K97, R101, K107, K111, R115, and K117 of the wild type mHCN1) may interact with 4,5-PIP₂. Facilitation of HCN channel gating may be effected by direct interaction between 4,5-PIP₂ and basic amino acid residues in the cytoplasmic region of the HCN channel. See hypothesis in the legend of FIG. 6.

### EXAMPLE

### 4-4βPMA Facilitation of HCN1 Channel Gating

Wild type mouse HCN1 was expressed in Xenopus oocytes. 2 MEV-clamp recordings were performed in the absence or following 10 or 30 minutes incubation in the presence of 200 nM 4βPMA, 200 nM 4αPMA, or the DMSO solvent alone. Cells were held at −30 mV. Immediately prior to the 3 s activation step a cell is stepped to +20 mV for 1 s to deactivate channels open at the holding potential and then stepped to the test potential. The results in FIG. 10 show that HCN channel gating is facilitated by 4βPMA, but not 4αPMA. CNBD-disabled HCN1 mutant, HCN1-R538E, was expressed in Xenopus oocytes. 2 MEV-clamp recordings were performed in the absence or following 10 or 30 minutes incubation in the presence of 200 nM 4βPMA, 200 nM 4αPMA, or the DMSO solvent alone. The results in FIG. 11 show that gating of HCN1-R538E is not altered by either adenylate cyclase activation or inhibition. These data indicates that 4βPMA facilitation of HCN1 channel gating is not mediated by cAMP.

HCN1 mutant, HCN1-REHR (H268R, R538E), was expressed in Xenopus oocytes. The two point mutations in HCN1-REHR disrupts the ability of the channel to couple with both the proton and the cAMP. 2 MEV-clamp recordings were performed in the absence or following 10 or 30 minutes incubation in the presence of 200 nM 4βPMA, 200 nM 4αPMA, or the DMSO solvent alone. The results in FIG. 12 show that 4βPMA facilitation of HCN1 channel gating is not mediated by protons.

Three vertebrate HCN channel isoforms, HCN1, HCN2, HCN4, and one invertebrate HCN channel isoform DmHCN(HCN channel of Drosophila melanogaster) were expressed in Xenopus oocytes and 2 MEV-clamp recordings were performed in the presence and absence of 200 nM 4βPMA. 200 nM 4βPMA facilitated the gating of each of these channels. See Table 2. The gating of these channels was unaffected by the inactive phorbol, 4αPMA or DMSO vehicle. The more distant but structurally related member of the hyperpolarization-activated ion channel family, the plant channel KAT1, was insensitive to 200 nM 4βPMA (See FIG. 14). This finding further reinforces our conclusion that the response of the HCN channels to DAG/4βPMA is a specific response involving selective coupling via a cascade involving a C1-binding protein.

TABLE 2
Conservation of 4βPMA response among HCN family members

| | WILD | | CA | | CA UNCOUPLED | |
|---|---|---|---|---|---|---|
| | TYPE | | UNCOUPLED | | H⁺ UNCOUPLED | |
| CHANNEL | ΔV½ | n | ΔV½ | n | ΔV½ | n |
| HCN1 | +15 ± 3 | 13 | +29 ± 3 | 5 | +32 ± 5 | 4 |
| HCN2 | +8 ± 2 | 18 | +15 ± 2 | 14 | +26 ± 3 | 8 |
| HCN4 | +17 ± 7 | 3 | | | | |
| DmHCN | +9 ± 4 | 3 | | | | |

Data are for n recordings after 30 min in 200 nM 4βPMA.

### EXAMPLE 5

### Diacylglycerol Facilitation of HCN Gating

CNBD-disabled channel mutant, HCN1-R538E, was expressed in Xenopus oocytes. 2MEV-clamp recordings were performed before or after perfusion of the cell with 30 μM R599492 or 59022 (both are DAG-kinase inhibitors) for 60 minutes, or DMSO for 60 minutes. The results in FIG. 13 show that R59949 and R59022 treatment facilitates HCN1-R538E activation. These data indicates that DAG may be involved in the facilitation of HCN1-R538E activation—a conclusion supported by the report that these treatments do double the DAG concentration in Xenoups oocytes during a 60 minute incubation.

### EXAMPLE 6

### The Core Region of HCN1 is Required for the 4βPMA Response

Wild type HN1, HCN1-R538E, HCN1-REHR, HCN1-ΔCα, HCN1-ΔCNBD, HCN1-ΔNvΔC, HCN1-ΔNvΔC-HR (ΔNvΔC mutation and the point mutation at position 268 from Histidine to Arginine) were expressed in Xenopus oocytes. 2 MEV-clamp recordings were performed following incubation in the absence and presence of 200 nM 4βPMA. The results in FIG. 15 show that the mutant channels have similar reaction to 4βPMA treatment as the wild type HCN channel, indicating that the core region, but not the N and C terminal variable regions nor the CNBD, is required for responding to 4βPMA.

Mutation constructs were made on the basis of HCN1-ΔNvΔC to produce mutations in all serine, threonine and tyrosine residues, which are potential phosphorylation sites. See FIG. 16A. The mutants were expressed in Xenopus oocytes. 2 MEV-clamp recordings were performed before or following 30 minutes incubation of 200 nM 4βPMA. The results in FIG. 16B show that 4βPMA facilitation of HCN channel gating is not affected by the mutations, suggesting that 4βPMA regulation of HCN channel gating does not require phosphorylation.

### EXAMPLE 7

### Modulation of HCN Activation Gating by the PTK Inhibitor Genistein

The effect of PTK inhibitor genistein on HCN channel activation was studies using wild type and CNBD-disabled HCN channels. Recordings were made in the presence and absence of genistein. The results suggest that HCN activation gating by the PTK inhibitor genistein is determined by nucleotide-occupancy of the cyclic nucleotide binding pocket of the channels.

FIGS. 17 A,C,E,G show the gating of wild type and CNBD-disabled HCN channels in the absence (top) and presence (bottom) of the PTK inhibitor, genistein (A: HCN1; C:HCN1-R538E; E: HCN2; G: HCN2-R538E).

FIGS. 17 B,D,F,H show the activation curves for each construct in the absence and presence of genistein, revealing that the weak facilitation of HCN1 by genistein (B) converts to a robust facilitation when the CNBD is disabled (D) (Right shifts shown in BLUE). In HCN2, the response to genistein converts from an inhibition (F) (left shift shown in RED) to facilitation (Right shift shown in BLUE) when the CNBD is disabled (H).

### EXAMPLE 8

### C-Linker is Required for Regulation of HCN Gating by the PTK Inhibitor

Activation of wild type HCN channels and mutant HCN channels with mutation in the C-linker or CNBD domains was recorded in the presence and absence of PTK inhibitor genistein. The results suggest that the HCN C-linker but not the CNBD is required for regulation of gating by the PTK inhibitor genistein.

The results in FIG. 18B show the effect of genistein treatment to activation of HCN1-R538E, HCN1-ΔCNBD and HCN1-ΔCterm. Left Panel: Change in V_{1/2} (V_{1/2} Final−V_{1/2} Initial) in control and genistein treated cells (90 μM for 30 min). Right Panel: Plot of Δ(ΔV1/2) shows genistein facilitation is independent of the CNBD (facilitation of HCN1-ΔCNBD and full length HCN1-R538E are the same) but requires the C-linker, as its deletion renders HCN1-ΔCterm insensitive to genistein.

FIG. 18C shows that genistein facilitation of cAMP insensitive HCN2 constructs is also independent of the CNBD.

While the foregoing invention has been described in some detail for purposes of clarity and understanding, it will be appreciated by one skilled in the art, from a reading of the disclosure, that various changes in form and detail can be made without departing from the true scope of the invention in the appended claims.

The following references are incorporated by reference herein in their entirety:
Anderson et al, 1999, J. Biol. Chem. 274: 9907-9910
Baggiolini et al, 1987, Exp. Cell Res. 169: 408-418
Cerbai et al, 2001, J. Mol. Cell Cardiol 33: 441-448
Chang et al, 1977, Nature 198: 1056
Chaplan et al, 2003, J. Neurosci 23: 1169-1178
Closse et al, 1981, J. Med. Chem. 24: 1465-1471
Cohen et al, 1972, Proc Natl Acad Sci USA 69: 2110
Fiers et al, 1973, Nature 273: 113
Fukami et al, 1988, Proc. Natl. Acad. Sci. USA 85: 9057-9061
Graham and van der Eb, 1978, Virology 54: 546
Gait, M. J. Ed, Oligonucleotide Synthesis, IRL Press, Oxford, 1984
Goeddel et al, 1980, Nucl Acids Res 8: 4057
Goulding et al, 1994, Nature 372: 369-374
Hammerling et al, Monoclonal Antibodies and T Cell Hybridomas, Elsevier, N.Y. 1981
Hsiao et al, 1979, Proc Natl Acad Sci USA 76: 3829
Karin et al, 1982, Nature 299: 797-802
Kohler et al, 1975, Nature 256: 495
Kohler et al, 1976, Eur. J. Immunol. 6: 511
Ludwig et al, 1998, Nature 393: 587-591
Matuoka et al, 1988, Science 239: 640-643
McCormick et al, 2001, Annu Rev Physiol 63: 815-846
Nolan et al, 2003, Cell 115: 551-564
Okada, T. et al, 1994, J. Biol. Chem. 269: 3563-3567
Ren et al, 1996, Mol. Biol. Cell 7: 435-442
Sambrook et al, Molecular Cloning, A Laboratory Manual, Cold Spring Harbor Press, Cold Spring Harbor, N.Y. 1989
Santoro et al, 1997, Proc. Natl. Acad. Sci. USA 94: 14815-14820
Santoro et al, 1998, Cell 93: 717-729
Schulze-Bahr et al, 2003, J. Clin. Invest. 111: 1537-45
Shimatake et al, 1981, Nature 292: 128
Stopkova et al, 2003, Am. J. Med. Genet. B. Neuropsychiatr. Genet. 123: 50-58
Stephens et al, 1989, Biochem. J. 259: 267-276
Stephens et al, 1991, Nature 351: 33-39
Stewart et al., Solid Phase Peptide Synthesis, 2nd ed., Pierce Chem. Co., Rockford, Ill. (1984) Tissue Cultures, Academic Press, Cruz and Patterson, Eds, (1973)
Van Solingen et al., J Bact (1977) 130: 946
Wainger et al., 2001, Nature 411: 805-810
Weernink et al, 2000, J. Biol. Chem. 275: 10168-10174
Whitman et al, 1988, Nature 332: 644-646
Wiesinger, D. et al, 1974, Experientia 30:135-136
Wigler et al., Cell (1979) 16: 777-785
Vlahos, C. J. et al, 1994, J. Biol. Chem. 269: 5241-5284
U.S. Pat. No. 6,703,485

What is claimed is:

1. A method of facilitating HCN channel activation in a cell comprising contacting a cell that expresses a HCN channel with an effective amount of a compound that increases the level of 4,5-PIP₂ in the cell.

2. The method of claim 1, wherein the compound is exogenous 4,5-PIP₂.

3. The method of claim 1, wherein the compound stimulates the activity of at least one kinase selected from the group consisting of PI4 kinase, PI5 kinase and Rho kinase.

4. The method of claim 3, wherein the compound is recombinant RhoA.

5. The method of claim 1, wherein the compound inhibits the activity of PI3 kinases.

6. The method of claim 5, wherein the compound is selected from the group consisting of wortmannin, bioflavenoid quercetin and LY294002.

7. The method of claim 1 further comprising contacting the cell with a second compound that increases the level of DAG and/or stimulates the activity of a C1-binding protein that mediates the activation of a HCN channel by DAG in the cell.

8. A method of inhibiting HCN channel activation in a cell comprising contacting a cell that expresses a HCN channel with an effective amount of a compound that decreases the level of 4,5-PIP₂ in the cell.

9. A method of claim 8, wherein the compound is an antibody binding specifically to 4,5-PIP₂.

10. The method of claim 8, wherein the compound is the monoclonal antibody KT10.

11. The method of claim 8 further comprising contacting the cell with a second compound that decreases the level of DAG and/or inhibits the activity of a C1-binding protein that mediates the activation of a HCN channel by DAG in the cell.

12. A method of screening for a compound that modulates the activation of a HCN channel comprising contacting a cell with the compound and determining the difference between the level of 4,5-PIP₂ in the presence of the compound and the level of 4,5-PIP₂ in the absence of the compound.

13. A compound that is capable of modulating the activation of a HCN channel identified according to the method of claim 12.

14. A method of treating or preventing a disease or condition in an individual in which there is an undesirable reduction of the activity of HCN channels comprising administering to such individual a pharmaceutically effective amount of a compound that increases the level of 4,5-PIP₂ in the individual.

15. The method of claim 14, wherein the compound is 4,5-PIP₂ or a pharmaceutically acceptable salt thereof.

16. The method of claim 14, wherein the compound is recombinant RhoA or a pharmaceutically acceptable derivative thereof.

17. A method of treating or preventing a disease or condition in an individual in which there is an undesirable elevation of the activity of HCN channels comprising administering to such individual a pharmaceutically effective amount of a compound that decreases the level of 4,5-PIP₂ in the individual.

18. The method of claim 17, wherein the compound is KT10 or a pharmaceutically acceptable derivative thereof.

19. A method of facilitating HCN channel activation in a cell comprising contacting a cell that expresses a HCN channel with an effective amount of a compound that increases the level of DAG in the cell.

20. The method of claim 19, wherein the compound is exogenous DAG.

21. The method of claim 19, wherein the compound an inhibitor of DAG-kinase activity.

22. The method of claim 21, wherein the compound is R59949.

23. A method of inhibiting HCN channel activation in a cell comprising contacting a cell that expresses a HCN channel with an effective amount of a compound that decreases the level of DAG in the cell.

24. A method of screening for a compound that modulates the activation of HCN channels comprising contacting a cell with the compound and determining the difference between the level of DAG in the presence of the compound and the level of DAG in the absence of the compound.

25. A compound identified by the method of claim 24, that is capable of modulating the activation of HCN channels.

26. A method of treating or preventing a disease or condition in an individual in which there is an undesirable reduction of the activity of HCN channels comprising administering to such individual a pharmaceutically effective amount of a compound that increases the level of DAG in the individual.

27. A method of treating or preventing a disease or condition in an individual in which there is an undesirable elevation of the activity of HCN channels comprising administering to such individual a pharmaceutically effective amount of a compound that decreases the level of DAG in the individual.

28. A method of facilitating HCN channel activation in a cell comprising contacting a cell that expresses a HCN channel with an effective amount of a compound that stimulates the activity of a C1-binding protein that mediates the activation of a HCN channel by DAG.

29. The method of claim 28, wherein the compound is 4βPMA.

30. A method of inhibiting HCN channel activation in a cell comprising contacting a cell that expresses a HCN channel with an effective amount of a compound that inhibits the activity of a C1-binding protein that mediates the activation of a HCN channel by DAG.

31. A method of treating or preventing a disease or condition in an individual in which there is an undesirable reduction of the activity of HCN channels comprising administering to such individual a pharmaceutically effective amount of a compound that stimulates the activity of a C1-binding protein that mediates the activation of a HCN channel by DAG in the individual.

32. A method of treating or preventing a disease or condition in an individual in which there is an undesirable elevation of the activity of HCN channels comprising administering to such individual a pharmaceutically effective amount of a compound that inhibits the activity of a C1-binding protein that mediates the activation of a HCN channel by DAG in the individual.

33. A method of inhibiting HCN channel activation in a cell comprising contacting a cell that expresses a HCN channel with an effective amount of a compound that inhibits the interaction between HCN channel and 4,5-PIP₂.

34. The method of claim 33, wherein the compound inhibits the interaction between one or more basic amino acid residues of the cytoplasmic region of the HCN channel and 4,5-PIP₂.

35. The method of claim 34, wherein the basic amino acid residues are located on the cytoplasmic portion of the core region of the HCN channel.

36. The method of claim 33, wherein the compound inhibits the interaction between the NT1 and NT2 regions of the HCN channel and 4,5-PIP₂.

37. The method of claim 33, wherein the compound is an antibody specifically binding to the HCN channel.

38. A method of treating or preventing a disease or condition in an individual in which there is an undesirable elevation of the activity of HCN channels comprising administering to such individual a pharmaceutically effective amount of a compound in the individual, wherein the compound inhibits the interaction between HCN channels and 4,5-PIP₂.

39. The method of claim 38, wherein the compound is an antibody that specifically binds to the NT1 and NT2 regions of the HCN channel.

40. A method of screening for a compound that modulates the interaction between HCN channels and 4,5-PIP₂ comprising contacting a cell that expresses a HCN channel with a test compound and 4,5-PIP₂ and measuring the gating activity of the HCN channel.
